(19) United States
(12) Patent Application Publication
(10) Pub. No.: US 2025/0356970 A1
(43) Pub. Date: Nov. 20, 2025

(54) KNOWLEDGE INFORMATICS AUTO REVIEWER FOR QUALITY CONTROL OF REPORTING

(71) Applicant: OmniSeq, Inc., Buffalo, NY (US)

(72) Inventors: Zachery BLISS, Buffalo, NY (US); Paul DEPIETRO, Buffalo, NY (US)

(73) Assignee: OmniSeq, Inc., Buffalo, NY (US)

(21) Appl. No.: 19/208,265

(22) Filed: May 14, 2025

Related U.S. Application Data
(60) Provisional application No. 63/647,572, filed on May 14, 2024.

(51) Int. Cl. G16H 15/00; G06F 16/23; G06F 16/2455; G06F 16/25; G16H 10/40

(52) U.S. Cl. CPC G16H 15/00; G06F 16/2365; G06F 16/2455; G06F 16/258; G16H 10/40

(57) ABSTRACT

An auto reviewer system designed to execute required rule checks, extract information of interest to human reviewers, and generate a final report with the results. The auto reviewer system operates by checking that an order satisfies a pre-defined set of conditions and raising a flag for each condition not satisfied. The flags considered pertinent to a user's understanding of the final report are then combined into order notes, and the order notes are automatically entered into the internal note fields of the corresponding orders in one or more workbenches. Additionally, flags raised that correspond to order issues requiring manual intervention trigger automatic support request notifications such as emails, which are sent to a user to be resolved. If an order has no issues requiring manual intervention, then the note generated for that order includes a complete statement and is passed for final report generation.

### CROSS-REFERENCE TO RELATED APPLICATION

The present application is a non-provisional application of and claims the benefit and priority under 35 U.S.C. 119(e) of U.S. Provisional Application No. 63/647,572, filed on May 14, 2024, the entire contents of which is incorporated herein by reference in its entirety for all purposes.

### FIELD

The present disclosure relates to laboratory test reporting, and in particular to a knowledge informatics auto reviewer system configured to ensure there are no defects present in healthcare reports and users such as healthcare providers have all the information necessary to communicate results accurately.

### BACKGROUND

Knowledge informatics is an interdisciplinary field dedicated to the systematic organization, management, and analysis of knowledge to facilitate decision-making, problem-solving, and innovation across various domains. By integrating principles of information science, data analytics, artificial intelligence, and knowledge management, it enables efficient access to actionable insights. This field is particularly important in environments that deal with vast amounts of complex data, such as healthcare, law, business, education, and research, where converting raw information into usable knowledge is essential.

The key components of knowledge informatics include knowledge representation, information retrieval, data integration, and analytics. Advanced methodologies like ontology and taxonomy development provide structured frameworks to classify and organize knowledge, promoting standardization and interoperability. Natural Language Processing (NLP) enhances information retrieval by enabling systems to analyze and understand unstructured data, while database management systems (DBMS) harmonize and manage data from diverse sources to ensure consistency and scalability. Additionally, semantic web technologies, such as RDF and OWL, facilitate machine-readable knowledge representation, and traditional statistical analysis supports evidence-based decision-making by identifying patterns and trends in data. Complementing these methods, data visualization techniques and data warehousing streamline the presentation and consolidation of complex datasets, while knowledge management systems (KMS) and collaborative filtering foster knowledge sharing and personalized resource recommendations. Together, these methodologies provide a comprehensive toolkit to efficiently organize, manage, and analyze knowledge across various domains.

Knowledge informatics practices are increasingly being integrated into healthcare reporting reviews to improve accuracy and reliability, ensuring providers receive error-free reports. One way this is done is through the integration of Electronic Health Record (EHR) data with data from various knowledge databases. EHRs include information, or data, related to patients such as patient demographics, medical history, medication records, immunization records, diagnostic and lab results, treatment plans, clinical notes, therapy considerations, appointment scheduling, insurance and billing information, and any other related information for patients. Knowledge databases offer healthcare providers real-time access to evidence-based guidelines, treatment protocols, clinical recommendations, current clinical interpretations of genetic testing results, and the like. Through this data integration, the healthcare reports generated are not only comprehensive but also aligned with the latest medical standards.

When integrating data from different sources for healthcare reporting, knowledge informatics tools like rule-based algorithms, can be implemented to automatically check and identify missing, inconsistent, duplicate, or incorrect data entries in reports before they are finalized. Additionally, implementation of structured frameworks, such as ontologies and taxonomies help ensure that diverse data sources, such as diagnostic results, treatment plans, genetic testing, and patient histories, are harmonized and accurately categorized within the EHR and knowledge databases systems. Consistency and accuracy in healthcare reporting is also significantly enhanced by the use of DBMS and NLP technologies, such as SQL queries, for efficient data management and retrieval of EHR data. For example, DBMS organizes EHR data into structured formats such as tables and schemas, enabling NLP algorithms to access and query data efficiently. On the other hand, knowledge databases tend to store raw data or have their data organized in data structures inconsistent with the data structures used by the EHR database manager. To facilitate data management and retrieval of data from knowledge databases, methods such as Extract, Transform, Harmonize (ETH) are used to ensure the efficient retrieval, standardization, and alignment of knowledge data with EHR data. These knowledge informatic practices promote standardization and interoperability during healthcare reporting review, assisting providers in identifying data discrepancies, such as missing or inconsistent data entries.

Another role for knowledge informatics in healthcare reporting includes the continuous updating of systems to incorporate new medical knowledge, regulatory changes, technological advancements, and updates to patient medical records, all of which is important for ensuring the accuracy, relevance, and reliability of healthcare reporting. Knowledge informatics systems, such as EHRs, Clinical Decision Support Systems (CDSS), and data management tools, are dynamic platforms that evolve alongside the healthcare industry. Regular updates ensure these systems reflect the latest clinical practices, research findings, and evidence-based guidelines, enabling healthcare providers to make informed decisions based on current standards of care. For example, as new treatments, diagnostic methods, or medications are developed, informatics systems are revised to include updated templates. This eliminates the risk of outdated information being used in reports, which could compromise patient care. By continuously refining informatics systems, healthcare facilities create an adaptive environment that supports high-quality, error-free reporting and ensures healthcare providers have access to the most current and reliable tools for patient care.

### BRIEF SUMMARY

Computer-program techniques are disclosed herein (e.g., a computer-implemented method, system and operations thereof, and non-transitory computer-readable medium storing code or instructions executable by one or more processors) to knowledge informatics auto reviewer system configured to ensure there are no defects present in healthcare reports and users such as healthcare providers have all the information necessary to communicate results accurately.

In some embodiments, a computer-implemented method comprises: determining that orders are available for review based at least on a status of each of the orders, wherein each of the orders comprise a series of data entries in a table, the status of each of the orders is associated with at least one of the data entries, a unique identifier for each of the orders is associated with at least one of the data entries, and the series of data entries are stored in a production level data store; generating a set of queries for each of the orders based on one or more query programming languages, wherein the set of queries for each of the orders comprise the unique identifier for the associated order; executing the set of queries for each of the orders on a database to retrieve data associated with each of the orders based on the unique identifier for each of the associated orders, wherein the data is replicated and maintained consistent with the production level data store that obtains at least some of the data from a laboratory instrument, a laboratory assay, a laboratory workstation, or any combination thereof; for each of the orders, analyzing the data associated with each of the orders and data retrieved from one or more other data stores based on rules defined in a configuration file, wherein the analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied; for each of the orders, processing the list of flagged conditions, wherein the processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof; and for each of the orders, generating, based on the series of data entries in the table and the processing the list of flagged conditions, a final report comprising a summary of the series of data entries.

In some embodiments, the computer-implemented method further comprises: extracting data from the production level data store, wherein the data is in non-standardized data files and comprises data expected to appear in the orders; transforming the data in the non-standardized data files into data in a standardized format using one or more transformation algorithms; and storing the data in the standardized form in the database, wherein the set of queries for each of the orders are executed on the data in the standardized format in the database to retrieve the data associated with each of the orders.

In some embodiments, the set of queries for each of the orders are executed on the database and the one or more other data stores to retrieve the data associated with each of the orders and the data retrieved from the one or more other data stores based on the unique identifier for each of the associated orders.

In some embodiments, the processing comprises correcting the information within the series of data entries associated with the order in the production level data store, and wherein the correcting comprises transmitting the list of flagged conditions associated with the one or more of the orders to a client device, and correcting, using the client device, the information within the series of data entries associated with the one or more of the orders in the production level data store based on the list of flagged conditions associated with the one or more of the orders.

In some embodiments, the processing comprises communicating the internal notes pertaining to the information within the series of data entries associated with the order to the production level data store, and wherein the communicating comprises generating the internal notes pertaining to the information within the series of data entries associated with the order based on the list of flagged conditions associated with the one or more of the orders, and transmitting one or more write requests to the production level data store for an internal notes field associated with the one or more orders.

In some embodiments, the processing comprises updating the status of one or more of the orders in the review log, and wherein the updating comprises writing the status of one or more of the orders in the review log based on the list of flagged conditions associated with the one or more of the orders, and transmitting the review log to a client device.

In some embodiments, the processing comprises sending one or more notifications to one or more users concerning one or more of the flagged conditions associated with one or more orders, and wherein the sending comprises generating one or more notification messages for the one or more of the flagged conditions associated with the one or more orders, and transmitting the one or more notification messages to one or more end points associated with the one or more users.

In some embodiments, a system is provided that includes one or more processors, and a memory that is coupled to the one or more processors and stores a plurality of instructions which, when executed by the one or more processors, cause the one or more processors to perform any of the methods disclosed herein.

In some embodiments, a computer-program product is provided that is tangibly embodied in a non-transitory computer-readable memory that includes instructions which, when executed by the one or more processors, cause the one or more processors to perform any of the methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a computing environment in accordance with various embodiments.

FIG. 2 illustrates an auto reviewer system in accordance with various embodiments.

FIG. 3 shows an example of a final report that is generated based on an error free order generated from the auto reviewer system described in FIG. 2 in accordance with various embodiments.

FIG. 4 shows a flowchart for reviewing reports using the auto reviewer system in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

Much of the healthcare workflow involves the generation and review of healthcare reports to aid healthcare professionals in the diagnosis of a disease or developing a treatment plan. Healthcare reports can include information on a patient's demographics (e.g., name, date of birth, gender, etc.), medical history, medications, vaccination records, laboratory and diagnostic test results, treatment plans, clinical notes, and so. Ensuring that the information that appears in a patient's health records is important for guaranteeing that they receive the best care possible. As such, health records are reviewed to identify potential reporting errors, such as a coding error or omission, making an outcome measure reportable, extracting laboratory values from free text to appropriately apply a clinical guideline, or identifying a clinical trial candidate. This process typically involves spending an exorbitant amount of manual review and resources to ensure accurate and high-quality reporting. Standard practice is for humans to manually review hundreds of complex reports one-at-a-time every day. Regardless of how intelligent, thorough, or simple the task is, the human mind is very bad at following rules consistently. Human execution of repetitive tasks frequently results in errors being made.

A key aspect of reviewing healthcare reports is verifying that the information presented is accurate, up-to-date, and aligns with current medical standards and knowledge. Medical reports often compile information from diverse sources, including internal organization databases and external public databases. Internal databases store information related to patients and organization specific testing results, while external public databases may provide data related to the clinical significance of genetic variants, the latest sequencing studies, and details on available clinical trials. Human reviewers must continuously monitor and cross-check these databases to confirm the accuracy and currency of the information presented in the reports. This process is fraught with challenges, including the difficulty of identifying subtle errors or inconsistencies, such as outdated timestamps, missing records, or mismatched values, particularly when dealing with large and complex datasets. Manual cross-checking is not only time-consuming but also prone to oversight, increasing the risk of human error. Furthermore, compliance with strict data privacy regulations like GDPR and HIPAA adds another layer of complexity, requiring reviewers to ensure sensitive information is handled securely and without unauthorized access. Communication gaps between teams responsible for updating and maintaining databases can exacerbate these difficulties, potentially resulting in incomplete updates or unclear documentation. These challenges underscore the need for computer assisted monitoring and maintenance of the databases from which reporting data is extracted to ensure healthcare reports meet the highest standards of accuracy and reliability.

In addition to verifying data consistency across various internal and external sources, the need to repeatedly access data from multiple systems significantly hinders reviewer efficiency. When a machine repeatedly accesses disparate databases, whether local or remote, each call consumes computational resources to establish connections, fetch data, and process information, leading to higher memory usage and increased latency. Remote calls exacerbate inefficiencies due to network delays and reliance on external systems, increasing the likelihood of connectivity issues and failed requests. Moreover, fragmented data across multiple systems requires frequent context switching and data transformation, which places additional strain on system memory and processing power. Consequently, machines spend considerable time managing communication protocols rather than focusing on core tasks, ultimately hindering scalability and responsiveness in data-heavy operations.

The above challenges are further compounded by the fact that each data entry that appears on a report often originates from databases with diverse formats and structures. Continuously retrieving non-standardized data from multiple sources places additional strain on computer systems, impacting memory usage, runtime efficiency, and the overall speed of manual document review. Non-standardized formats require extra processing to interpret, transform, and reconcile data, which increases computational overhead and prolongs runtime. Parsing and converting these disparate formats demand additional memory allocation, further straining system resources, especially when handling large datasets. For example, within an organization, different departments often use distinct file formats to manage their databases. Medical imaging departments commonly rely on DICOMs to store and transmit X-rays, MRIs, CT scans, and ultrasound images, while pathology images may be stored as TIFF, SVS, JPEG, or PNG files. Other data entries, such as clinical notes or appointment schedules, may be stored in JSON or XML formats. Tabular data, like billing records or lab results, are often managed through CSV files, while documents such as consent forms or compliance reports are stored in PDF format. Externally managed databases, such as genomic databases, frequently use FASTA files to store nucleotide or amino acid sequences and VCF files to capture genetic variations. The coexistence of these varied formats amplifies the challenges associated with accessing, integrating, and ensuring the accuracy of healthcare report data and the overall review process.

To address these challenges and others, disclosed herein are computer implemented methods, systems, and computer-program products that extract pertinent result information for reporting and automatically reviews orders for defects. Described herein is an auto reviewer system designed to facilitate the review of orders prior to being converted into reports for review by healthcare professionals. The auto reviewer system overcomes challenges faced by conventional report reviewing practices by implementing one or more state maintenance methods to ensure that updates made to remote or external databases are synchronized with a local database accessed by the auto reviewer system. Additionally, the computing environment that the auto reviewer system operates within utilizes a local database that stores relevant data entries extracted from the remote/external databases in a standardized structure (e.g., a tabular structure). This approach eliminates the need to repeatedly access data from multiple systems, significantly reducing computational resource demands and improving overall system efficiency. Moreover, by storing data in a standardized format, the software minimizes computational overhead and runtime by avoiding the continuous transformation of non-standardized raw data files.

The auto reviewer system confirms that an order satisfies a pre-defined set of conditions and raises flags for each condition not satisfied. Further, the auto reviewer system automatically extracts information considered by healthcare professionals to be the most critical to ensure the order and final report correctly conveys results and corresponding knowledge to the healthcare professional for treating their patient. The flags considered pertinent to a healthcare professional's understanding of the report content of each order are combined into order notes, and the order notes are automatically entered into the internal note fields of the corresponding orders. Additionally, flags raised that correspond to order issues requiring manual intervention trigger automatic support request emails sent to the reviewer. The flags considered pertinent to a healthcare professional's understanding and the issues requiring manual intervention are defined by configuration files within the auto reviewer system project directory. If an order has no issues requiring manual intervention, then the note generated for that order includes the statement “Review Complete”. The notes and support requests generated for each order are uploaded to a database which are viewed by a sign-out team.

Compared to standard practices of human reviewers, implementation of the auto reviewer system and techniques described herein has reduced the time required to complete review of medical orders by over 90%, has reduced the amount of energy and focus required to complete reviews by at least 95%, and has reduced the number of critical defects missed during review by 100%. Moreover, compared to conventional order/report review systems, implementation of the auto reviewer system and techniques described herein has improved CPU performance due to factors like more efficient use of memory and other resources (e.g., CPU processing cycles), and more efficient instruction execution, leading to faster processing and improved multitasking. Additionally, these advancements in use of memory and other resources, and the execution of instructions, have significantly reduced latency and improved overall system responsiveness.

In some aspects, a computer-implemented method comprises: determining that orders are available for review based at least on a status of each of the orders, wherein each of the orders comprise a series of data entries in a table, the status of each of the orders is associated with at least one of the data entries, a unique identifier for each of the orders is associated with at least one of the data entries, and the series of data entries are stored in a production level data store; generating a set of queries for each of the orders based on one or more query programming languages, wherein the set of queries for each of the orders comprise the unique identifier for the associated order; executing the set of queries for each of the orders on a database to retrieve data associated with each of the orders based on the unique identifier for each of the associated orders, wherein the data is replicated and maintained consistent with the production level data store that obtains at least some of the data from a laboratory instrument, a laboratory assay, a laboratory workstation, or any combination thereof; for each of the orders, analyzing the data associated with each of the orders and data retrieved from one or more other data stores based on rules defined in a configuration file, wherein the analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied; for each of the orders, processing the list of flagged conditions, wherein the processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof; and for each of the orders, generating, based on the series of data entries in the table and the processing the list of flagged conditions, a final report comprising a summary of the series of data entries.

As used herein, the terms “about,” “similarly,” “substantially,” and “approximately” are defined as being largely but not necessarily wholly what is specified (and include wholly what is specified) as understood by one of ordinary skill in the art. In any disclosed embodiment, the term “about,” “similarly,” “substantially,” or “approximately” may be substituted with “within [a percentage] of” what is specified, where the percentage includes 0.1 percent, 1 percent, 5 percent, and 10 percent, etc.

As used herein, when an action is “based on” something, this means the action is based at least in part on at least a part of the something.

### Computing Environment

Certain processes and methods described herein are performed within a computing environment comprising a computer, microprocessor, software, module, other machines such as sequencers, or combinations thereof. The methods described herein typically are computer-implemented methods, and one or more portions or steps of the method are performed by one or more processors (e.g., microprocessors), computers, systems, apparatuses, or machines (e.g., microprocessor-controlled machine). Computers, systems, apparatuses, machines, and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

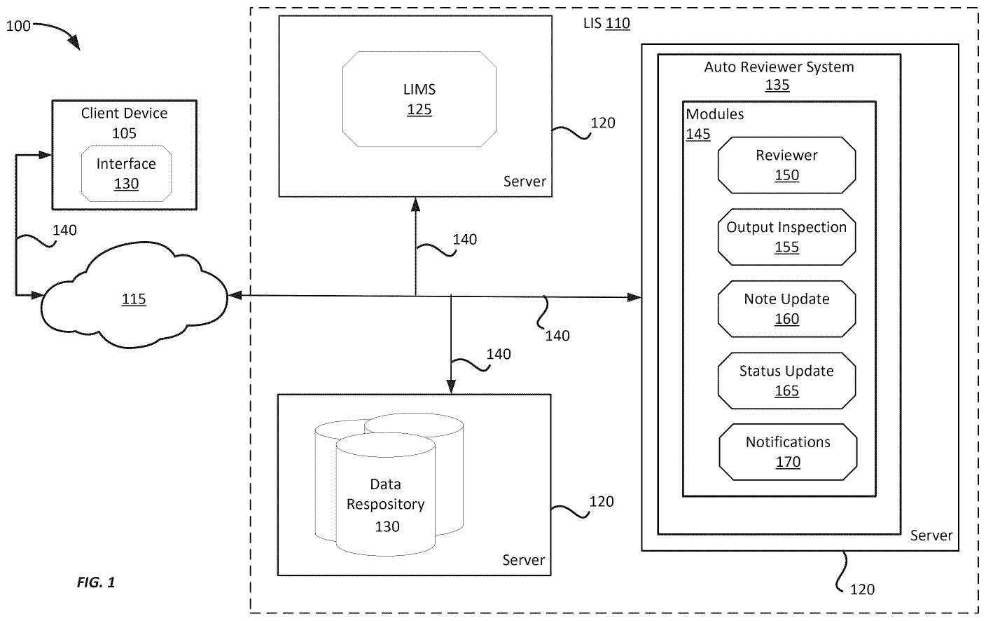

FIG. 1 shows a computing environment 100 in accordance with aspects of the present disclosure. Computing environment 100 is only one example of a suitable computing environment and is not intended to suggest any limitation as to the scope of use or functionality of the systems, methods, and data structures described herein. Neither should computing environment 100 be interpreted as having any dependency or requirement relating to any one or combination of components illustrated in computing environment 100. A subset of systems, methods, and data structures shown in FIG. 1 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like

Computing environment 100 includes a client device 105 and a Laboratory Information System (LIS) 110 connected to each other by a network 115. LIS 110 serves as a centralized system of hardware, firmware, and software for handling laboratory data, workflows, and processes, enabling a laboratory to efficiently manage patient information, test orders, sample tracking, and results reporting. LIS 110 includes servers 120 that provide various resources, data, services, or programs for other computers (e.g., other servers or client device 105) over network 115. In the instance of computing environment 100, the servers are shown to be providing key aspects of the present disclosure, which includes a Laboratory Information Management System (LIMS) 125, one or more data stores or repositories 130, and an auto reviewer system 135. However, it should be understood that the servers 120 could provide additional or other resources, data, services, or programs such as Human Resource Information Systems (HRIS), mail transfer and delivery agents, other application services, other data management or database services, and the like.

Although FIG. 1 illustrates a particular arrangement of a client device 105 and a LIS 110, this disclosure contemplates any suitable arrangement of a client device 105 and a LIS 110. As an example, and not by way of limitation, two or more client devices 105, a data repository 130, and auto reviewer system 135 may be connected to each other directly, bypassing network 120. As another example, two or more client devices 105, a data repository 130, and an auto reviewer system 135 may be physically or logically co-located with each other in whole or in part. Moreover, although FIG. 1 illustrates a particular number of a client device 105, a data repository 130, an auto reviewer system 135, and network 115, this disclosure contemplates any suitable number of client devices 105, data repositories 130, auto reviewer systems 135, and networks 115. As an example, and not by way of limitation, computing environment 100 may include multiple client devices 105, data repositories 130, auto reviewer systems 135, and networks 115.

A client device 105 is an electronic device including hardware, software, or embedded logic components or a combination of two or more such components and capable of interacting with LISM 125, data repository 130, and auto reviewer system 135 with respect to analyzing laboratory data including healthcare orders and reports and ensuring healthcare providers have all the information necessary to communicate results accurately in accordance with techniques of the disclosure. The client device 105 may be a computing device such as a conventional computer, a distributed computer, or any other type of computer (e.g., portable handheld devices, general purpose computers such as personal computers and laptops, workstation computers, wearable devices, gaming systems, thin clients, various messaging devices, sensors or other sensing devices, and the like). The computing device may execute and run various types and versions of software applications and systems (e.g., Internet-related apps, communication applications (e.g., E-mail applications, short message service (SMS) applications), LIMS applications, auto review system 135) and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®) using one or more communication protocols. Portable handheld devices may include cellular phones, smartphones, (e.g., an iPhone), tablets (e.g., iPad®), personal digital assistants (PDAs), and the like. Wearable devices may include Google Glass® head mounted display, and other devices.

In some aspects, the client device 105 includes a processing unit, a system memory, and a system bus that operatively couples various system components including the system memory to the processing unit. There may be only one or there may be more than one processing unit, such that the processor of computing device includes a single central-processing unit (CPU), or a plurality of processing units, commonly referred to as a parallel processing environment. The system bus may be any of several types of bus structures including a memory bus or memory controller, a peripheral bus, and a local bus using any of a variety of bus architectures. The system memory may also be referred to as simply the memory and includes read only memory (ROM) and random access memory (RAM). A basic input/output system (BIOS), containing the basic routines that help to transfer information between elements within the computing device, such as during start-up, is stored in ROM. The computing device may further include a hard disk drive for reading from and writing to a hard disk, a magnetic disk drive for reading from or writing to a removable magnetic disk, and an optical disk drive for reading from or writing to a removable optical disk such as a CD ROM or other optical media.

The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer-readable instructions, data structures, program modules and other data for the client device 105. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk, optical disk, ROM, or RAM, including an operating system, one or more application programs, other program modules, and program data. A user may enter commands and information into the client device 105 through input devices such as a keyboard and pointing device (e.g., mouse). Other input devices may include a microphone, joystick, game pad, satellite dish, scanner, or the like. These and other input devices are often connected to the processing unit through a serial port interface that is coupled to the system bus, but may be connected by other interfaces, such as a parallel port, game port, or a universal serial bus (USB). A monitor or other type of display device is also connected to the system bus via an interface, such as a video adapter. In addition to the monitor, computing devices typically include other peripheral output devices, such as speakers and printers.

LIS 110 is platform designed to manage and streamline the operations of medical and clinical laboratories. It serves as a centralized system for handling laboratory data, workflows, and processes, enabling laboratories to efficiently manage patient information, test orders, sample tracking, and results reporting. LIS 110 is important in ensuring the accuracy, traceability, and timeliness of laboratory activities, which are used for providing reliable diagnostic services. In some aspects LIS 110 integrates with other healthcare systems, such as Electronic Health Records (EHRs), to facilitate seamless communication between the laboratory and healthcare providers. Additionally, LIS 110 supports compliance with regulatory standards by automating documentation and maintaining data integrity, making it useful in clinical and research environments.

The architecture of LIS 110 is comprised of a client-server or cloud-based model, where users interact with the system through front-end interfaces (e.g., client device 105) while the back-end handles data storage, processing, and analytics. Hardware components of LIS 110 includes servers (including servers 120) for storing and managing data, workstations for laboratory users, and interfaces for connecting laboratory instruments to the system. On the software side, LIS 110 includes modules for sample management, test workflow configuration, result validation, and reporting (including auto reviewer system 135). Integration tools, such as APIs, may be used for connecting the LIS 105 with external systems like EHRs and billing platforms. Security features, including encryption and access controls, are used to ensure the confidentiality and integrity of sensitive laboratory and patient data. In some aspects, LIS 110 may also incorporate advanced analytics, bioinformatics, and machine learning tools to improve efficiency and decision-making in laboratory operations.

Network 115 can be any type of network familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 115 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 140 may connect a client device 105, LIMS 125, data repositories 130, and auto reviewer system 135 to network 115 or to each other. This disclosure contemplates any suitable links 140. In particular embodiments, one or more links 140 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 140 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 140, or a combination of two or more such links 140. Links 140 need not necessarily be the same throughout a computing environment 100. One or more first links 140 may differ in one or more respects from one or more second links 140.

Servers 120 are computers or computing devices designed to manage, store, process, and deliver data or services (e.g., data and services related to LIMS 125 and auto reviewer system 135) to other devices, such as client 105, over network 115. The servers 120 can be physical machines or virtual instances (e.g., virtual machines) created through virtualization technologies. The servers 120 operate using specialized hardware, such as high-performance CPUs, large amounts of RAM, and storage arrays, to handle intensive workloads. They are equipped with server-grade operating systems (e.g., Windows Server, Linux-based distributions) and software that facilitate specific functions, such as hosting websites, managing databases, running applications, or handling email services. To provide data or services such as those related to LIMS 125 and auto reviewer system 135, servers 120 listen for incoming requests from clients (e.g., client device 105) via network protocols (e.g., HTTP for web services, SMTP for email, or FTP for file transfers). When a request is received, a server processes it using its resources and returns the appropriate response, such as delivering lab results, analyzing lab orders and reports, and generating reports.

Servers 120 can also support multi-user environments, enabling simultaneous access to shared resources, and can be integrated into a distributed system or cloud infrastructure to ensure scalability, reliability, and high availability. For example, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers 120, storage devices such as data repositories 130, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS users may access resources and services through network 115 and use the cloud provider's services to install the remaining elements of an application stack. For example, the user can log in to the IaaS platform to create virtual machines such as servers 120, install operating systems (OSs) on each virtual machine, deploy middleware including databases such as those that may be included in data repositories 130, create storage buckets for workloads and backups, and even install enterprise software such as LIMS 125 and auto review system 135 into one or more virtual machines. Users can then use the provider's services to perform various functions, including troubleshooting laboratory equipment issues, monitoring performance of laboratory equipment, reviewing laboratory orders, results, and reports, generating reports, analyzing laboratory results, generating laboratory results, etc.

LIMS 125 is a software-based solution that supports various laboratory operations including managing, automating, and optimizing laboratory workflows, data, and resources across various operational domains. LIMS 125 is integrated with laboratory instruments, workbenches, third-party software, and enterprise systems to centralize laboratory operations while maintaining traceability and data integrity. Through data management and workflow automation, the LIMS 125 enhances efficiency, reduces manual errors, and enables scalability in the laboratory environment such as computing environment 100.

LIMS 125 implementation within the computing environment 100 involves a combination of hardware, software, and network resources. At the core of the implementation is the client-server or cloud-based architecture, where the LIMS software resides on centralized servers or in distributed cloud environments (e.g., one or more of servers 120—physical or virtual machines). Servers 120 are equipped with high-performance computing resources and database management systems (e.g., SQL, NoSQL databases) to store and process laboratory data. LIMS 125 interfaces with laboratory instruments through middleware or direct integration using APIs, or instrument control software to enable automated data acquisition. Laboratory users interact with the LIMS 125 through client device interfaces accessible on workstations, tablets, or mobile devices (e.g., client device 105), while administrators configure workflows and access controls to tailor the system to laboratory-specific requirements. For compliance, LIMS 125 incorporates audit trails, electronic signatures, and regulatory reporting tools, ensuring adherence to standards such as ISO 17025, GLP, or FDA 21 CFR Part 11. Advanced analytics, machine learning algorithms, and data visualization tools may also be included to provide actionable insights, optimize operations, and support decision-making within the laboratory ecosystem.

A data repository such as one of the data repositories 130 is a centralized location for storing, managing, and maintaining data. It functions as a digital warehouse that enables the efficient organization, retrieval, and analysis of data. Data repositories 130 are used for systems such as LIMS 125 and auto review system 135 that require the storage of structured, semi-structured, or unstructured data. The data repositories 130 are characterized by their storage infrastructure, which can include on-premise servers, cloud-based systems, or hybrid solutions. They include access control mechanisms to ensure secure data management, supporting various formats such as relational, semi-structured, or unstructured data. Scalability is a key attribute, allowing data repositories 130 to handle growing data volumes, while integration capabilities enable seamless connection with external systems and data pipelines. Querying tools, such as SQL, APIs, or other interfaces, are used to facilitate efficient data retrieval and manipulation. Although the data repositories 130 are shown on the same server in FIG. 1 it should be understood that they could be spread across multiple servers in various configurations without departing from the spirit and scope of the present disclosure.

In some instances, a data repository such as one of the data repositories 130 is a database which is a specialized form of data store designed to manage structured data efficiently. While all databases are data stores, not all data stores are databases. For example, in other instances, a data repository such as one of the data repositories 130 is a data store which includes various systems for storing data, such as file systems, key-value stores, and object stores. This broad category covers any technology used to persist data, whether structured, semi-structured, or unstructured. A client device 105 may interact with a data store through a structured process that involves communication over a network using APIs, query languages, or other protocols. The client device 105 sends requests to the data store to perform operations such as retrieving, updating, inserting, or deleting data. These requests may be formatted in a query language (e.g., SQL for relational databases) or through API calls (e.g., REST or GraphQL for web-based systems). The data store, equipped with access control mechanisms, authenticates the client and verifies permissions before executing the requested operations. Once processed, the data store returns the requested data or a confirmation of the operation's success back to the client device, for example in a structured format such as JSON or XML for easy parsing and utilization. This interaction is governed by network protocols such as HTTP or HTTPS and optimized for efficiency, scalability, and security, ensuring that the data exchange is reliable and compliant with organizational or legal standards.

In some aspects, one or more of the data repositories 130 are used to store data and other information for use by client device 105, LIMS 125, and/or auto reviewer system 135. For example, a first data repository 130 may be a data store used to store data and information from the LIMS 125 in a first format (e.g., JSON files). In some instances, the data and information relates to testing of samples (e.g., gene definitions, current and reporting symbols, aliases, mapping to various tests and components within tests, etc.), to processing of samples (e.g., sample identifiers, laboratory steps performed, sample yields, sequencer well placement, slides remaining after testing, patient information, etc.), to demographics of samples, and/or to all the raw data files used as source information for generating a report. A second data repository 130 may be a database used to store data and information in a second format (e.g., entries in a database table) to be used as input into the auto reviewer system 135 for analyzing orders. In some instances, the data and information stored in the second data repository 130 is a subset of the data and information stored in the first data repository 130.

The auto reviewer system 135 comprises a set of tools and software modules 145 that ensure accurate and high-quality generation of reports. The auto reviewer system 135 operates by checking that an order satisfies a pre-defined set of conditions and raises flags for each condition not satisfied. In the configuration depicted in FIG. 1, the set of tools and software modules 145 are implemented using five primary modules (i.e., a self-contained piece of code that performs a specific function within a larger software program that may be executed by one or more processors, hardware components, or combinations thereof): a reviewer module 150, an output inspection module 155, a notes update module 160, a status update module 165, and a notifications module 170. The auto reviewer system 135 may reside in a variety of locations including servers 120. For example, an auto reviewer system 135 executed by server 120 may be local to server 120 or may be remote from server 120 (e.g., on a virtual machine) and in communication with server 120 via a network-based or dedicated connection of network 115.

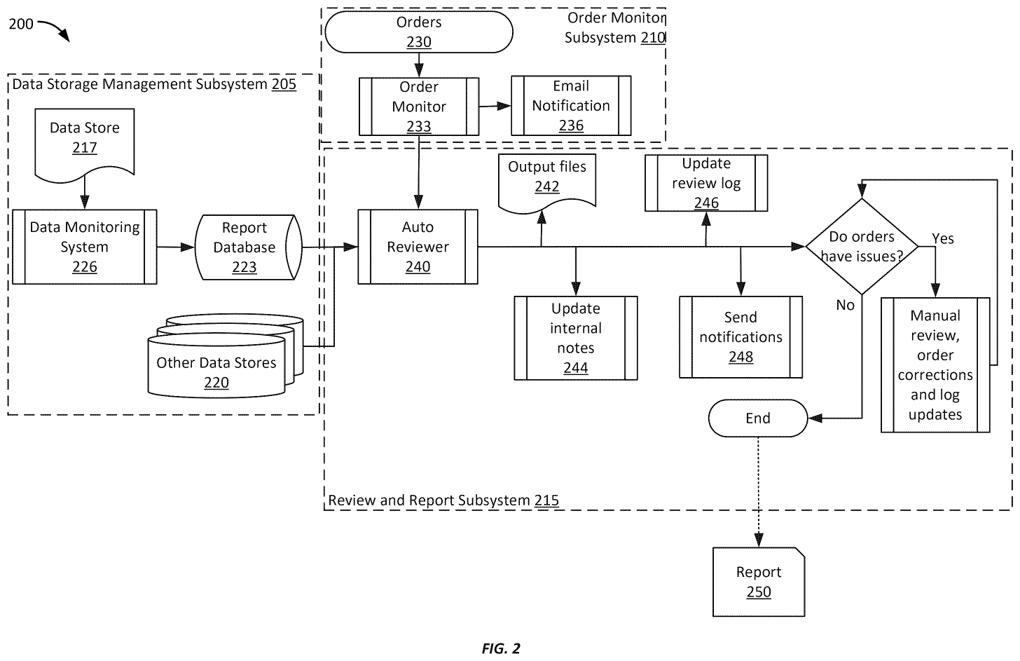

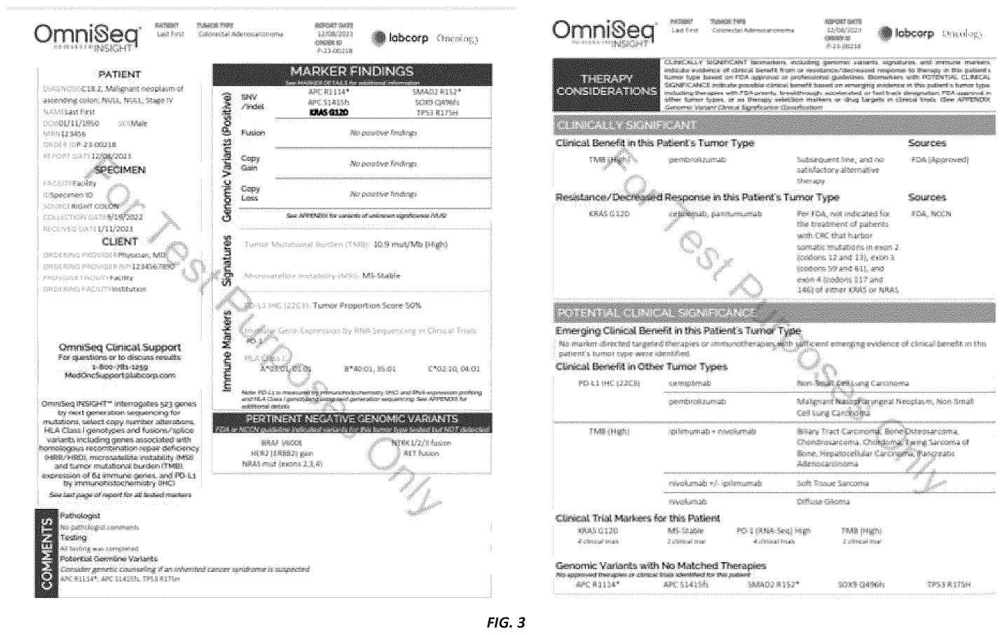

As discussed in detail below with respect to FIG. 2, reviewer module 150 is responsible for analysis of an order, including: cross-referencing the reported data in an order to the corresponding raw or original data files (stored in data repositories 130), extracting relevant results, and delivering output files to a specified end point (e.g., email address) for inspection. The output files may include one or more error flags for data that may require inspection via the output inspection module 155. Error flags raised by reviewer module 150 and inspected using the output inspection module 155 may cause correction of the data in the order and generation of notes via the notes update module 160, and the corrected order can be rerun by reviewer module 150 for further analysis. If error flags are still reported, the process of inspection and correction may be repeated until no more error flags are reported. Once an order no longer contains error flags, the status update module 165 can be used to update the status of the order, complete the order as an error free order, and generate notifications via notifications module 170. The error free order can then be used by the auto reviewer system 135 or another system such as the LIMS 125 (e.g., one or more workbenches) to generate a final report (see, FIG. 3).

### Auto Reviewer System

FIG. 2 depicts a simplified block diagram for an auto reviewer system 200 configured to extract relevant data points that are expected to appear in an order and perform quality control steps, using a rule-based approach, to ensure no data reporting defects appear on a final report. The auto reviewer system 200 may be implemented using software only, hardware only, firmware only, or any combination of hardware, software, and/or firmware. The auto reviewer system 200 (auto reviewer system 135 described with respect to FIG. 1) is run in a computing environment (computing environment 100 described with respect to FIG. 1) and a user may execute the auto reviewer system 200 on a computing system such as a client device (client device 105 described in FIG. 1). Execution of the auto reviewer system 200, alone or in combination with other systems and/or software program such as the part of the LIMS or one or more workbenches, causes performance of one or more processes and/or programs as discussed in detail below. It should be understood that the one or more processes and/or programs can be executed or performed as part of an automated process, a semi-automated process, a manual process, or any combination thereof. Moreover, it should be understood that the one or more processes and/or programs can be executed as part of an iterative process that ultimately generates one or more final reports. An iteration or an iterative process being the process of repeating a set of instructions or steps multiple times or cycles. For example, a set of instructions or steps may be executed for generating one or more error free orders or final reports and repeatedly executing the set of instructions or steps multiple times or over multiple cycles results in the processing of one or more orders to generate one or more error free orders or final reports. Each cycle of the set of instructions or steps may be executed serially, or multiple cycles of the set of instructions or steps may be executed in parallel.

As depicted in FIG. 2, the auto reviewer system 200 comprises a data storage management subsystem 205 to acquire and store data expected to appear in an order, an order monitoring system 210 to determine when one or more orders are ready for review, and a review and report subsystem 215 to review order data, perform one or more actions based on the review, and ultimately trigger the generation of a final laboratory report. This setup can allow the auto reviewer system 200 to deliver real-time, responsive interactions while ensuring high availability, security, and performance scalability to meet varying demand levels. Although FIG. 2 provides an example of the auto reviewer system 200 being directed towards laboratory or healthcare related reports, one of ordinary skill in the art can appreciate how the techniques and methods described herein can be applied to the review of other reports, datasets, and systems through code adaptation without expanding the scope of the present disclosure.

The data storage management subsystem 205 comprises various data repositories (e.g., the data repositories 130 described with respect to FIG. 1) including a data store 217, one or more other data stores 220, and a report database 223. The data storage management subsystem 205 further comprises a data monitoring system 226 configured to monitor and ensure the data in report database 223 is up to date, i.e., consistent, with data stored in data store 217.

Data store 217 and the other data stores 220 function as repositories for storing, managing, and organizing raw laboratory data from diverse sources (e.g., various applications and systems associated with LIMS 125 described with respect to FIG. 1) using a variety of data storage solutions, including databases, file systems (e.g., CSV files, JSON files), cloud storage (e.g., Amazon S3, Google Cloud Storage), and distributed systems. Examples of raw laboratory data that can be stored in data store 217 and the other data stores 220 include, without limitation, data related to a patient, a specimen, a client, biomarker (e.g., genomic or immunological) findings, therapy considerations, clinical significance, comments, and other relevant information generated in a laboratory as part of performing one or more clinical assays. Notably, each of these data types may have their own unique data format file type, meaning the data stored in data store 217 and the other data stores 220 is in a non-standardized or non-uniform format. For example, medical images widely use DICOM files to store and transmit X-rays, MRIs, CT scans, and ultrasound images. With respect to genomics data, files such as FASTA, which contain nucleotide or amino acid sequences, and VCF, which records genetic variations, are used. Other file types may include JSON or XML files for structured data like clinical notes or appointment schedules, CSV files for tabular data such as billing records or lab results, and PDFs for documents, consent forms, or compliance reports.

In some aspects, one or more workbenches are implemented on one or more servers (e.g., servers 120 described with respect to FIG. 1) and connected to data store 217 and/or and the other data stores 220. The one or more workbenches are platforms configured to provide an end-to-end analysis solution for next generation sequencing (NGS) and non-NGS data (e.g., data from other laboratory assays) that integrate directly with lab systems such as the LIMS, automates clinical interpretation (e.g., somatic and germline variant interpretation), matches patients to personalized therapy and clinical trial options, and generates custom reports. The flexible and customizable one or more workbenches enable users to go from order entry to quality control to clinical interpretation and reporting, quickly and easily. For example, with respect to germline testing, one of the workbenches can ingest germline VCF files derived from one or more analysis platforms, quickly annotate variants with various resources, and provide streamlined support for variant interpretation and reporting on findings.

The data generated by the one or more workbenches, e.g., VCF files, annotated variants, classifications, patient data, result reports, and the like, can be stored in data store 217 and/or the other data stores 220 as one or more file types (e.g., JSON files) and communicated to other laboratory applications and systems such as the LIMS and auto reviewer system 200. In some aspects, a workbench is used for processing orders (e.g., includes reviewing the order, selecting and implementing workflows for running assays on the order, and obtaining results of the analytical assays), which generates a series of data entries for each order, e.g., VCF files, annotated variants, classifications, patient data, result reports, and the like. The series of data entries for each order are maintained on the data store 217 and retrievable for use by the auto reviewer system 200 for reviewing orders, as described in detail below. In these respects, it should be understood that data store 217 and the other data stores 220 are to be considered part of a production level system (a software or hardware system that is designed, implemented, and maintained to be used in a real-world, operational environment such as a clinical laboratory environment).

In order for the auto reviewer system 200 to utilize these diverse data types, it leverages an Extract, Transform, Harmonize (ETH) data management process to ensure the efficient retrieval, standardization, and alignment of data for seamless use across various systems and applications. Initially, ETH begins with an extract phase, which involves retrieving the unstructured, raw data from data store 217. In some instances, data store 217 is a file system storing non-standardized raw data files as JSON files. To improve data retrieval, the auto reviewer system 200 extracts the data expected to appear in an order from the data store 217, transforms the extracted data into a standardized format and stores the standardized data on the report database 223. The transformation phase of ETH includes applying one or more transformation algorithms to clean, normalize, and standardize the raw data through processes such as removing duplicates, mapping fields to schemas, and converting data types. In some instances, the raw JSON files may be parsed and transformed from their semi-structured state by flattening, where the JSON data is mapped into rows and columns so that the JSON data are stored as tables on the report database 223. Finally, the harmonize phase ensures consistency and interoperability by aligning data from different sources with unified naming conventions, formats, and semantics, resolving discrepancies and enabling integration. This can include linking all the data belonging to one patient from different sources with a unique ID (e.g., a patient ID) or to one order from different sources with a unique ID (e.g., an order ID).

Advantageously, performing ETH on the data (e.g., JSON files) in data store 217 to transform them into data entries in report database 223 (e.g., relational database) reduces the amount of data being stored in memory at the report database 223 by optimizing the data structure and eliminating redundancy. JSON files often contain nested, hierarchical data with repeated keys and unstructured information. During the Extract phase, relevant data is extracted from the raw JSON files (data expected in to be in orders, e.g., the series of data entries corresponding to each order), filtering out unnecessary or unused data. The Transform phase involves restructuring and normalizing the extracted data into a format compatible with report database 223. This includes breaking down nested structures into flat tables, eliminating redundant data, and converting complex data types into simpler, space-efficient formats. The Harmonize phase ensures data consistency by standardizing formats, deduplicating entries, and consolidating related records, reducing storage overhead. By transforming the data into a schema specific to report database 223, the database can utilize indexing, normalization, and relationships to store data efficiently, avoiding the need to hold large, unstructured JSON files in memory. As a result, the report database 223 only stores the essential, structured data, significantly reducing memory usage compared to loading and processing raw JSON files directly. In so doing, the ETH method drastically improves the performance of the auto reviewer system 200 by reducing the amount of data needing to be stored in local memory and minimizing the consumption of computational and network resources on data store 217 at the production level.

By nature, laboratory data is fluid and electronic health record (EHR) data, laboratory assay results, and scientific advancements are continuously being generated or updated. Examples of this can include new data or updates pertaining to a patient's EHR record or laboratory assay results, a user's (e.g., pathologist's) interpretation of the assay results, gene names or the clinical significance of a genetic variant being updated as a result of scientific discovery, clinical research findings impacting an assay, and the like. To ensure the data stored in data store 217, and subsequently the data extracted and stored in the report database 223, reflects the most current and accurate information, the data monitoring system 226 is used to ensure replication and consistency between the two data stores.

The data monitoring system 226 leverages various mechanisms to ensure data replication and consistency between data store 217 and the report database 223 such as synchronization mechanisms, event-driven architecture, and/or real-time tracking. For example, when there are changes (e.g., data creation, update, or deletion) made to the raw data files in the data store 217, an event-driven mechanism is triggered to capture and process the event so that the relevant data is updated in the report database 223. Moreover, the data monitoring system 226 can use techniques like timestamping to compare the timestamps of data entries in the data store 217 against the timestamps of the data entries in the report database 223 to ensure data consistency. Replication and consistency between the two data stores using timestamps may be achieved through conflict resolution mechanisms and replication protocols. When data is updated in data store 217, each update is assigned a unique timestamp, based on the system's local clock or a globally synchronized clock (e.g., via NTP). During replication, the timestamps are used to determine the order of updates and resolve conflicts in case of concurrent writes. For example, in last-write-wins (LWW) conflict resolution, the update with the most recent timestamp is applied to ensure consistency. Vector clocks or logical clocks can be used in the instance of a distributed system to capture causality between operations, allowing the system to determine whether updates are dependent or concurrent. Replication protocols such as log-based replication rely on timestamps to sequentially propagate changes to replicas, ensuring that updates are applied in the correct order to the report database 223. Periodic reconciliation processes may compare timestamps across data store 217 and report database 223 to detect and resolve inconsistencies. Combined with techniques like snapshot isolation and transactional guarantees, timestamp-based replication enables consistent data synchronization while managing conflicts efficiently.

Clinical laboratories utilize various other data stores (such as the other data stores 220), including Clarity and Connect, along with other systems like LabKey Sample Manager, Knowledge Databases, Fluics, EMR/EHR, and LIMS. These systems help manage samples, data, and workflows, ensuring efficient and accurate data handling within clinical settings. The other data stores 220 can be of various types of data stores such as relational, time-series, graph, hierarchical, columnar, spatial, and the like configured for storing electronic laboratory data. Flexible electronic laboratory data in the other data stores 220 have many advantages. For example, users can view, sort, pool, and appropriately route laboratory information to better support trend analyses, clinical decision making, and clinical charting including for use with aspects of auto reviewer system 200. More specifically, in the context of the auto reviewer system 200, the data stored in the other data stores 220 are used for various aspects including order monitoring and cross-referencing with data stored in database 223 during the review of orders, as described in further detail below.

In some instances, at least one of the other data stores 220 is a knowledge database such as a genomics database storing genetic information. For example, information pertaining to gene names, gene reporting symbols, formal and/or common naming conventions for genes, gene aliases, etc. may be stored. Additionally, DNA/RNA sequencing results (e.g., sequencing reads, sequence mapping results, genetic variants, gene fusions), biomarkers, and data linking genes to specific diseases are stored. The genomics database can also include mappings between organization specific internal references and external references. For example, an external reference can include a list of target genes needing to be sequenced to check for variants which are mapped to an internal reference of available gene panels the organization can perform. Additionally, data related to biomarker findings may also be stored. Biomarker findings include information related to genomic variants that tested positive, genomic signatures (e.g., tumor mutation burden, microsatellite instability, etc.), positive immune markers (e.g., PD-L1 immunohistochemistry results, immune gene expression assessed by RNA sequencing, HLA Class, etc.), and pertinent negative genomic variants (e.g., genomic variants known to be associated with a specific tumor type but were not detected).

In some instances, at least one of the other data stores 220 is a sample processing database that stores data related to how a patient sample is processed, what assays have or still need to be conducted and so on. A sample processing database may store its data as a table, where the rows correspond to a patient, sample, or order via a unique identifier (e.g., order ID) and the columns represent different procedures and/or results. Examples of column data may include order ID, sample ID, laboratory assays performed, how much sample material was used, experimental yields, well placement, how much sample remains after testing, sample type (e.g., tissue slide or block), purpose (e.g., quality control, testing, and the like), tissue quality control determination (e.g., pass or fail), the facility where a specimen was collected/processed, the source of a specimen (e.g., left/right colon), specimen collection date, the date a specimen is received, and the like.

In some instances, at least one of the other data stores 220 is a patient database that stores data related to patient demographics (e.g., age, sex, income level, race, employment, location, homeownership, level of education, etc.), patient information and health history (e.g., address, prior assays results, weight, blood pressure, etc.), test orders (e.g., in progress, completed, etc.), the order IDs, report data, and any other information that is related to the patient such as diagnoses, treatment plans, prescribed medications, immunization records, allergies. Additionally, patient database may also include testing appointment history, billing information, and insurance details. These records are designed to provide healthcare providers and laboratories with an integrated view of a patient's health, facilitating informed decision-making, coordinated care, and enhanced patient outcomes.

The order monitoring system 210 is used by the auto reviewer system 200 to detect when orders 230 become available for execution by the auto reviewer 240 and execute the auto reviewer 240 to review/analyze the orders 230. The general order intake and monitoring process starts with receipt of one or more laboratory orders for performance of laboratory assays on specimens associated with patients. In some instances, one order, two orders, or batches of orders (e.g., comprising more than two orders) are received. The orders 230 are processed by at least a portion of the LIMS (e.g., a workbench), which transforms the unstructured form of the original orders into a series of entries in a table of data store 217 (e.g., one or more of the repositories 130 described with respect to FIG. 1). The series of entries in the table are essentially the reformatted original order and referred to hereafter as the orders 230. The series of entries for each order are associated with a unique order identifier and one or more of the entries indicate the status (e.g., waiting, pending, complete) of the order. Assay protocols are executed on specimens using workbenches and laboratory instruments (e.g., sequencers) in accordance with the information in the orders (e.g., information on assays to be run for each order and specimen). As assay protocols are executed and one or more components of each assay are completed, users (e.g., laboratory personnel such as laboratory technicians) review and validate the results of the one or more components of each assay. When assays are performed in accordance with quality control and assurance and the results are validated, the users or automated systems update one or more of the entries in the table that indicate the status (e.g., waiting, pending, ready for review, complete, etc.) of each component of each assay performed for an order.

The order monitor 233 monitors (e.g., via polling) the entries in the table that indicate the status (e.g., waiting, pending, ready for review, complete, etc.) of each component of each assay. The entries in the table that indicate the status of each component of each assay that the order monitor 233 is monitoring are located in at least one of the other data stores 220 (alternatively the entries being monitored may be stored in data store 217 and thus also present in report database 223). Polling or a polled operation denotes the process of repeatedly sampling the status of the entries in the table that indicate the status of each component of each assay. This process can occur hundreds to thousands of times a second. When the status of all or a subset of the components of each assay are ready for review (this can be preconfigured), the order monitor 233 sends a notification (e.g., email notification 236) to a user, software program (e.g., auto reviewer 240), and/or a system/subsystem that indicates which associated orders are ready for review by the auto reviewer 240.

The review and report subsystem 215 comprises the auto reviewer 240 and additional software modules (e.g., output inspection module 155, notes update module 160, status update module 165, and notifications module 170 described with respect to FIG. 1) for processing orders 230 determined to be ready for review by the order monitor 233. Specifically, the auto reviewer 240 is software designed to facilitate the review of orders 230 prior to user sign-out and generation of a final report. Auto reviewer 240 operates by checking that an order satisfies a pre-defined set of conditions and raising a flag for each condition not satisfied. The flags considered pertinent to user's (e.g., pathologist's) understanding of the final report content of each order are then combined into order notes, and the order notes are automatically entered into the internal note fields of the corresponding orders in one or more workbenches (on the production side). Additionally, flags raised that correspond to order issues requiring manual intervention trigger automatic support request notifications such as emails, which are sent to a user such as a reviewer. The flags considered pertinent to a user's (e.g., pathologist's) understanding and the issues requiring manual intervention are defined by configuration files within the project directory of the auto reviewer 240. If an order has no issues requiring manual intervention, then the note generated by the auto reviewer 240 for that order includes a complete statement such as “KI AutoReview Complete” and is passed for final report generation. The notes and support request notifications generated for each order are uploaded to a data store (e.g., data store 217), which can be viewed by one or more users (e.g., a sign-out team) and the sign-out status of the review process for each order can be updated through a dashboard of one or more workbenches.

In general, the basic operation and steps taken to complete review of orders using the review and report subsystem 215 and a description of how to complete each step are as follows:
1. Run the auto reviewer 240;
2. Inspect the output generated by the auto reviewer 240;
3. Correct the orders requiring manual or automated intervention;
4. Update the sign-out status via the dashboard to indicate the orders requiring intervention are complete; and
5. Communicate the output summary of the auto reviewer 240 to one or more users and/or system (e.g., the sign-out team).

In order to execute and run auto reviewer 240 in a computing environment (see FIG. 1 for additional details), a user may access the program by navigating to a link within an application or workbench and initiate the program (e.g., clicking on a user interface element). In some instances, the user may be prompted to enter one or more user authentications (e.g., password) prior to or upon initiating the program. In some instances, the program associated with the auto reviewer 240 can be initiated automatically, e.g., when orders are identified as being ready for review. In some aspects, the auto reviewer 240 can be initiated with parameters that change its operation. The parameters and their effects include:
orders: The orders parameter allows a user to select the orders to be reviewed (i.e., the orders identified by order monitor 233 that are ready for review). For example, to run the auto reviewer 240 on orders order_id_1 and order_id_2, the run command may be: sudo python3 driver.py -o “order_id_1, order_id_2”.
environment: The environment parameter allows a user to select the server (e.g., server01, sever-test01, and server02) on which orders should be reviewed. For example, to review orders on server01, the run command may be: sudo python3 driver.py -e “server01”. The default value may be set as “prod” leading to the default behavior of orders being reviewed on the production server-server01.
development: The development parameter allows a user to run the auto reviewer 240 for development purposes only, meaning that the auto reviewer 240 will not send support requests or upload results to the signout_status and signout_support_requests tables. For example, the run command may be: sudo python3 driver.py -d.
metadata: the metadata parameter allows a user to run the auto reviewer 240 without first confirming that one or more order files have been updated on the date of the run and that one or more data stores have been updated in the last predetermined number (e.g., 15) minutes. For example, the run command may be: sudo python3 driver.py -m. The default behavior of the auto reviewer 240 is to cancel the review if the one or more order files have not been updated on the day of the run or the one or more data stores have not been updated in the last predetermined number (e.g., 15) minutes.

When the auto reviewer 240 is executed, the operating system loads the reviewer program's executable file from storage (e.g., hard drive or SSD) into the computer's RAM, preparing it for execution. The CPU then fetches the reviewer program's instructions (e.g., code written in a computer programming language) from RAM, decodes each instruction to determine the required action, and executes them, which may involve calculations, data transfers, or interactions with input/output devices. During execution, the reviewer program may access system resources such as files, network connections, or hardware components, with the operating system managing these interactions to ensure proper permissions and resource allocation. Outputs or changes made by the reviewer program are stored in memory or written to storage as needed, such as saving files or updating databases. This cycle of fetching, decoding, and executing instructions continues sequentially (or in parallel) until the reviewer program completes its tasks. Finally, the reviewer program signals its termination to the operating system, which then releases the allocated resources, including memory and processor time, to ensure system efficiency.

As part of execution of the auto reviewer 240, a configuration file including predefined settings, parameters, and options is loaded and provides the instructions for how the auto reviewer 240 should process each order (i.e., the orders identified by order monitor 233 that are ready for review and/or orders specifically included in the orders parameter). The configuration file may be written in formats such as JSON, XML, YAML, or INI, and store key-value pairs, structured data, or plain text instructions. When the auto reviewer 240 is run, the configuration file is parsed into its individual parts (i.e., tasks) by a parser—a specialized tool designed to process specific file formats. The parser reads the file line by line or section by section, identifies the structure of the data, and converts it into a format compatible for the auto reviewer 240, such as variables or objects in memory. Once parsed, the auto reviewer 240 uses the extracted settings to execute tasks (e.g., rules) based on the configuration file's instructions. The configuration file can include at least 10 tasks, at least 20 tasks, at least 30 tasks, at least 40 tasks, at least 50 tasks, at least 75 tasks, at least 100 tasks, at least 150 tasks, at least 200 tasks, or more tasks that are customizable for the type of order being processed by the auto reviewer 240. In some instances, the configuration file includes at least 150 or more tasks.

In some instances, the tasks in the configuration file have been developed by gathering input from human report reviewers and healthcare professionals. Human reviewers provided insights into potential issues they have experienced or could foresee in various report sections, while healthcare professionals identified the result information they consider important for inclusion in a report. These responses are then translated into a comprehensive set of reporting rule requirements and information extraction targets, forming the basis for the tasks and rules outlined in the configuration file. In other instances, artificial intelligence and machine learning techniques can be applied to generate the tasks included in the configuration file. By analyzing large datasets to identify patterns and trends, artificial intelligence and machine learning approaches can learn the information needed to create the tasks. Machine learning models such as large language models (LLMs) can automate the extraction of relevant information, optimize rule generation, and continuously refine the rules based on new data or feedback, ensuring the auto reviewer 240 adapts to evolving requirements and improves accuracy over time.

A nonlimiting example of a configuration file that may be used by the auto reviewer 240 is shown below in Example 1. The example configuration file may be written in the XML format where each row represents a task (e.g., a rule) that is executed by the auto reviewer 240 during cross-referencing. In this example, each task is a rule that is checked for every order 230. In some instances, the tasks include rules that when flagged indicate that a particular rule has been broken, a data entry in the order is not consistent with the data entry in the report database 223, a data entry value is null, and so on. In other instances, some tasks may be logic conditions where if the task occurs, a notification (e.g., an internal note) is made. For example, a patient may have repeat orders (e.g., an initial order for a first test and subsequent orders for follow-up tests) and when this occurs, the task is to include a note in the final report that the patient has multiple results for the same test.

As shown in Example 1, each task (e.g., rule) includes several predefined settings including Module, Field, Flag, Hold_Flag, Note_Flag, email_address, email_subject, and email_text. The Module, Field, and Flag fields indicate which rule is being referenced. The Hold_Flag field indicates whether an order should be held for manual review or not when the rule is broken. The Note_Flag field indicates whether an internal note should be entered into a corresponding data entry filed in the data store 217 or not when the rule is broken. And finally, the email_address, email_subject, and email_text fields indicate where the support request notification (in this instance an email) should be sent, what the subject of the email should be, and what the text of the email should be when an order is held for manual review. For example, for order ID 1, the flag “Distance is missing” is raised in the Trials field of the actionability tab. Per the instructions of the configuration file, the auto reviewer 240 marks the order for hold based on the “Hold Flag” and generates a note based on the “Note Flag.” Further, the configuration file indicates who should receive the note (via the email address filed), what the subject of the email should be (via the email subject field) and what the email text should read (via the email text field).

### Example 1: Configuration File

| | | | | Hold | Note | | Email | |
|---|---|---|---|---|---|---|---|---|
| Module | Field | Flag | Action | Flag | Flag | Email Address | Subject | Email Text |
| Actionability | Trials | Distances | Check | 1 | 1 | name@email.com | Trial | This order |
| tab | | missing | zip | | | | distance | is missing |
| | | | | | | | missing | trial |
| | | | | | | | | geolocation. |
| | | | | | | | | Please |
| | | | | | | | | investigate |
| | | | | | | | | and correct. |
| Actionability | Last match | No | Run | 1 | 1 | name@email.com | No | This order |
| tab | date | MATCH | MATCH | | | | MATCH | has a |
| | | date | | | | | date | missing |
| | | | | | | | | MATCH |
| | | | | | | | | date. Please |
| | | | | | | | | investigate |
| | | | | | | | | and correct. |
| Actionability | Association | Novel | Check | 0 | 0 | name@email.com | | |
| tab | | association | value | | | | | |
| Actionability | Association | RNA | | 1 | 1 | name@email.com | RNA | This order |
| tab | | expression | | | | | expression | has an RNA |
| | | in clinical | | | | | in clinical | expression |
| | | benefit | | | | | benefit | association |
| | | section | | | | | section | in the |
| | | | | | | | | clinical |
| | | | | | | | | benefit |
| | | | | | | | | section. |
| Amendment | Amendment | Null reason | | 1 | 1 | name@email.com | Null reason | This order |
| reason section | reason | in complete | | | | | in complete | has a null |
| | | amendment | | | | | amendment | amendment |
| | | | | | | | | reason. |
| | | | | | | | | Please |
| | | | | | | | | investigate |
| | | | | | | | | and correct. |
| Amendment | Amendment | Null reason | | 1 | 1 | name@email.com | Null reason | This order |
| reason section | reason | in open | | | | | in open | has a null |
| | | amendment | | | | | amendment | amendment |
| | | | | | | | | reason. |
| | | | | | | | | Please |
| | | | | | | | | investigate |
| | | | | | | | | and correct. |
| Amendment | Amendment | Null | | 0 | 0 | name@email.com | Null | |
| reason section | reason | pathologist | | | | | pathologist | |
| | | comment in | | | | | comment in | |
| | | open | | | | | open | |
| | | amendment | | | | | amendment | |
| Appendix | Variants of | Null | Check to | 0 | 0 | name@email.com | | |
| section | unknown | | s . . . | | | | | |
| | significance | | | | | | | |
| Appendix | Intermediate | Null | | 0 | 0 | name@email.com | | |
| section | findings | | | | | | | |
| | with | | | | | | | |
| | potential | | | | | | | |
| | clinical | | | | | | | |
| | support | | | | | | | |
| Appendix | Variants of | Potential | | 0 | 1 | name@email.com | | |
| section | unknown | MET exon | | | | | | |
| | significance | 14 skip | | | | | | |
| | | present | | | | | | |
| . . . | | | | | | | | |

As part of execution of the auto reviewer 240, the auto reviewer 240 processes, based on the tasks or rules in the configuration file, each order (i.e., the orders identified by order monitor 233 that are ready for review and/or orders specifically included in the orders parameter) by comparing (cross-referencing) the series of table entries associated with each order (via order identifier) in the report database 223 to the data in the other data stores 220 (via order identifier).

In order to perform the comparing, the corresponding data in the report database 223 and other data stores 220 are retrieved by executing queries on the report database 223 and other data stores 220. For example, report database 223 may be a relational database and each table of report database 223 may contain rows (records) and columns (fields), with each column representing a specific attribute of the data and each row representing a unique entity or instance. The tables in a relational database are linked through primary keys (unique identifiers for rows within a table) and foreign keys (references to primary keys in other tables), enabling efficient organization and retrieval of related data. Relational databases are managed using relational database management systems (RDBMS) to manage and interact with report database 223. RDBMS uses query languages such as structured query language (SQL) as the primary means of interacting with the data in report database 223. SQL allows users to retrieve, insert, update, and delete data by writing commands that specify the desired operations. For example, a query can extract data from one or more tables using SELECT statements, with filtering criteria such as WHERE clauses to narrow results. Relational databases may also support JOIN operations, enabling users to combine data from multiple tables based on their relationships. Advanced SQL features, like aggregation (GROUP BY, SUM, COUNT) and sorting (ORDER BY), can also be used to analyze and present data in meaningful ways. By leveraging the relational structure and SQL capabilities, report database 230 provides a powerful and flexible framework for managing and querying large datasets efficiently. As should be understood, report database 230 may be queried using one or more other types of query language such as SPARQL, XQuery, and Cypher. Moreover, it should also be understood, that it is contemplated that some or all of the other data stores 220 are not relational databases and it is further contemplated that other types of query language such as GraphQL, SPARQL, XQuery, NoSQL, Cypher, DMX, MDX, and the like may be used for querying the other data stores 220.

In some instances, report database 230 and the other data stores 220 are associated in such a way that the data corresponding to a particular order are labeled with a primary key-a unique order identifier, e.g., order ID number. As such, queries may be formatted to extract the data entries expected to appear in any given order where only the data entry corresponding to the unique order identifier needs to be identified. For example, preconfigured queries (e.g., SQL queries) can be populated with the unique order identifier and executed on the report database 223 and other data stores 220 to extract query results for any given order.

Once the corresponding data in the report database 223 and other data stores 220 are retrieved, the auto reviewer 240 processes the data based on the tasks or rules in the configuration file. More specifically, the auto reviewer 240 compares (cross-references) the series of table entries associated with each order in the report database 223 to the corresponding data in the other data stores 220 based on the tasks or rules in the configuration file. This process triggers a series of automatic steps performed by the various modules of the review and report subsystem 215 based on the outcome of the compares.

The first automatic step is the generation of output files 242. The output files 242 may be in various formats known in the art, for example, and without limitation, excel format. In some instances, the output files 242 include a first table that lists conditions flagged by the auto reviewer 240 during order cross-referencing. See Example 2.

### Example 2: Rules Flagged by the Reviewer Program for a Batch of Orders

| Order ID | Module | Field | Flag |
|---|---|---|---|
| 1 | Markers with potential | Emerging clinical benefit in | Emerging clinical benefit in |
| | clinical significance section | this patient's tumor type | this patient's tumor type null |
| 2 | Clinically significant | Resistance/decreased | Resistance/decreased |
| | markers section | response in this patient's | response in this patient's |
| | | tumor type | tumor type null |
| 3 | Marker details section | Fusion | Fusion no results |
| 4 | Marker details section | CNV | CNV no results |
| 5 | Client section | Ordering facility | Lilly/HCA case |
| 6 | SNV tab | Alteration | Potential miscall: |
| | | | HSP90AA1 K363del, |
| | | | vaf = 0.4659, |
| | | | pos = 102551276; HSP90AA1 |
| | | | K363E, vaf = 0.0536, |
| | | | pos = 102551278 |
| 7 | Markers with potential | Emerging clinical benefit in | Emerging clinical benefit in |
| | clinical significance section | this patient's tumor type | this patient's tumor type null |
| 8 | Clinically significant | Resistance/decreased | Resistance/decreased |
| | markers section | response in this patient's | response in this patient's |
| | | tumor type | tumor type null |
| 9 | Marker details section | Fusion | Fusion no results |
| 10 | Marker details section | CNV | CNV no results |
| 11 | Markers with potential | Clinical benefit in other | Clinical benefit in other |
| | clinical significance section | tumor types | tumor types null |
| 12 | Marker details section | Fusion | Fusion no results |
| 13 | Marker details section | CNV | CNV no results |
| 14 | Client section | Ordering provider | Null provider suffix |
| 15 | Immunotherapy targets with | Targets with trials | Zero matches non-zero |
| | trials section | | matchable: [‘NECTIN2’, |
| | | | ‘VSIR’, ‘MAGEA4’, |
| | | | ‘CD68’, ‘PVR’, ‘CD40’, |
| | | | ‘CD4’, ‘CD163’, ‘CSF1R’, |
| | | | ‘MX1’, ‘CD86’, ‘TGFB1’, |
| | | | ‘CXCR2’, ‘TLR8’] |
| . . . | | | |

In some instances, the output files 242 include a second table showing the consolidated results for each order that are then communicated to a user. See Example 3. Result consolidation involves transforming the output (e.g., the Flag field in Example 2) from the auto reviewer 240 into understandable natural language (e.g., internal note) for the user. In some instances, the transformation process is done using a “Note Flag” field in a configuration file, see Example 1 above. When the “Note Flag” field is indicated (e.g., indicated by a 1 in Example 1) for a particular task, the auto reviewer 240 fills in variable slots of a predefined note structure, provided by the configuration file, with the information provided in the cross-referenced data entries for the order to generate the internal note.

### Example 3: Internal Summary Table

| Order | |
|---|---|
| ID | Internal Note |
| 1 | AutoReview Complete |
| 2 | Potential miscall: HSP90AA1 K363del, vaf = 0.4659, pos = |
| | 102551276; HSP90AA1 K363E, vaf = 0.0536, pos = 102551278 |
| | HOLD. |
| 3 | AutoReview Complete |
| 4 | AutoReview Complete |
| 5 | 2 matchable filtered mutations with low vaf: PBRM1 E1189fs |
| | vaf = 0.0213; PTPN11 N58H vaf = 0.0114. AutoReview Complete |
| 6 | AutoReview Complete |
| 7 | Matchable filtered mutation with low vaf: ARID1A Q1425* vaf = |
| | 0.0164. AutoReview Complete |
| 8 | Potential miscall: PMS2 L729fs, vaf = 0.3068, pos = 6018315; |
| | PMS2 T728A, vat = 0.284, pos = 6018320 HOLD. Matchable |
| | filtered mutation with low vaf: SMARCA4 c.5008-2A > C vaf = |
| | 0.0193 |
| 9 | Matchable filtered mutation with low vaf: TP53 E336* vaf = |
| | 0.0305. AutoReview Complete |

In some instances, the output files 242 include a third table that identifies support requests that require manual or automated intervention to correct inconsistencies between data in the order 230 (i.e., the report database 223) and data in the other data stores 220. See Example 4.

### Example 4: Summary of Support Request Requiring Manual Intervention

| | | | Support Email | Support Email | |
|---|---|---|---|---|---|
| Order ID | Flag | Date | Address | Subject | Resolved Flag |
| 1 | Potential miscall | 2024 May 6 | name@email.com | Potential miscalls | 0 |
| 2 | PDL1 score null | 2024 May 6 | name@email.com | Null PD-L1 assay | 0 |
| 3 | PD-L1 assay | 2024 May 6 | name@email.com | PD-L1 assay | 0 |
| | selections | | | selections missing | |
| 4 | PD-L1 assay | 2024 May 6 | name@email.com | PD-L1 static fields | 0 |
| | selections | | | missing | |
| 5 | Indeterminate | 2024 May 6 | name@email.com | Indeterminate | 0 |
| | pertinent negative | | | pertinent negative | |
| 6 | TMB final decision | 2024 May 6 | name@email.com | TMB decision | 0 |
| | incorrect | | | incorrect | |
| 7 | HLA final decision | 2024 May 6 | name@email.com | HLA decision | 0 |
| | incorrect | | | incorrect | |
| 8 | PD-L1 assay | 2024 May 6 | name@email.com | PD-L1 static fields | 0 |
| | selections | | | missing | |

The second automatic step is updating internal notes 244. As described above with respect to Example 2, when the “Note Flag” field is indicated for a particular task, an internal note is generated corresponding to the task. The generated internal note may be automatically communicated with and stored in the data store 217 with corresponding order data using one or more APIs (e.g., see links 140 with respect to FIG. 1). When the auto reviewer 240 does not flag any task, the internal note indicates the review is complete (shown as AutoReview Complete in Example 3). The third automatic step performed by the auto reviewer 240 is updating review log 246 to indicate the status of an order. For example, after review and report subsystem 215 has completed one cycle of order review, the status of one or more orders may be updated from “ready to review” to “complete” or “ready to review” to “hold” or “hold” to “complete”. The fourth automatic step is sending a notification 248 such as an email notification that delivers the output files 242 to a specified end point such as an email address provided in the configuration file.

For those orders with a status indicating a data reporting defect is present (e.g., a status of “hold” or “needs reviewed”), manual intervention may be required to correct the defect. The reports requiring manual intervention are split into two categories: orders with issues reviewer personnel (e.g., an individual trained to review and correct orders) can correct themselves, and orders with issues that can only be corrected by a support team member (e.g., a lab scientists, pathologists, etc.). Orders with issues the reviewer personnel can correct themselves are corrected and the reviewer manually updates the internal notes 244 and review log 246. For orders with issues that can only be corrected by a support team member, the reviewer personnel forwards the email to the relevant support team member for correction. Corrections are made by a user (e.g., reviewer personnel or support team member) using a part of the LIMS or one or more workbenches to access one or more data entries of the series of data entries for the corresponding order in the data store 217 and input the correction to the data within the one or more data entries of the series of data entries. The correction is then stored/saved in the data store 217 and replicated in the report database 223.

Nonlimiting examples of issues that require a support team member to correct are listed and described below:
potential miscalls: This support request is issued if an alteration (e.g., genomic variant) is suspected to be a potential miscall. The potential miscall support request is unique in the sense that instead of being order-specific, a single email covering the potential miscalls across all orders is generated. Upon notification of requesting correction, the support team inspects the alterations and reply with interpretations of the potential miscalls. The reviewer then manually enters the interpretations into the corresponding internal note fields, then updates the review log to “complete” (assuming this was the only support request related to the order).
indeterminate pertinent negative: This support request is issued if a component of a pertinent negative is included in the Indeterminate Wildtype list. The reviewer overrides the relevant pertinent negative(s) to Unknown, updates the internal note to indicate that the pertinent negative was overwritten, and saves the correction. By way of example, and not limitation, a reviewer may receive an email indicating the pertinent negative(s) corresponding to the NRAS Exon 2, NRAS Exon 3, and NRAS Exon 4 mutations should be overridden and updated to Unknown. The internal note includes “overrode NRAS exon 2, NRAS exon 3, and NRAS exon 4 pertinent negatives due to indeterminates” and the correction is saved.
pertinent negative removed without associated T1A therapy: This support request is issued if an alteration is removed from the Pertinent Negatives section without a corresponding T1A therapy surfacing in the Therapy Considerations section. The reviewer inspects the report in the report database 223 and determines why the corresponding T1A therapy is not surfacing. If the pertinent negative was removed without an associated T1A therapy surfacing because the pertinent negative is overly general and the T1A requires a specific mutation, then the reviewer updates the internal note to include a comment explaining that reasoning. If the pertinent negative was removed without an associated T1A therapy surfacing for an unknown reason, then the support request forwards the email to the appropriate personnel for further review.
missing pertinent negative: This support request is issued if one or more of the pertinent negatives associated with the report disease name is not included in the report even though the relevant test components passed. The reviewer inspects the order in the report database 223 and determines if there is any context which explains why the missing pertinent negative is valid. If no relevant context is found, the reviewer forwards the email to the appropriate personnel for correction. Once the pertinent negatives are corrected, the reviewer deletes the missing pertinent negative flag from the internal note and completes the order.
null pertinent negatives: This support request is issued if the pertinent negatives section is null even though the relevant test components passed. The reviewer inspects the order in the report database 223 to determine if there is any context which explains why the section is null. For example, if MATCH has not been run for the order. If running MATCH does not resolve the problem and there is no other context suggesting the null pertinent negatives makes sense, then the reviewer forwards the email to GenomeOncology to resolve. Once the null pertinent negatives are resolved, the reviewer deletes the null pertinent negatives flag from the internal note and completes the order.
alteration from inactive test mode present: This support request is issued if the report includes alterations that correspond to an inactive test mode. The reviewer inspects the report in the report database 223, confirms that the alterations are present on the report, performs one of the remove the alterations, forwards the support request notification such as an email to laboratory personnel or forwards the support request notification to administrative personnel depending on the situation. If the alterations are removed during the updating of the review log 246, then the reviewer removes them. If the alterations are not removed during the updating of the review log 246 but can be removed during the genomic analysts review step, the reviewer forwards the notification to laboratory personnel. If the alterations are not removed during either the updating of the review log 246 or the genomic analysts review step, the reviewer forwards the email to the appropriate personnel to resolve.
HLA decision incorrect: This support request is issued if the HLA decision is incorrect based on the Biomarker component status and DNA metric results. The reviewer inspects the report and the internal note in the report database 223 and, if there is no context in the internal note 244 to justify the decision, the reviewer forwards the email to appropriate personnel to resolve. If the sign-out support team confirms that the incorrect decision is unintentional, then the reviewer corrects the decision. If the sign-out support team provides context that implies the incorrect decision is intentional, then the reviewer does nothing.
TMB decision incorrect: This support request is issued if the TMB decision is incorrect based on the biomarker component status and DNA metric results. The reviewer inspects the report and the internal note 244. If there is no context in the internal note to justify the decision, the reviewer emails the appropriate personnel to resolve. If the sign-out support team confirms that the incorrect decision is unintentional, then the reviewer corrects the decision. If the sign-out support team provides context that implies the incorrect decision is intentional, the reviewer does nothing.
HLA results missing: This support request is issued if HLA results are missing from the report. The reviewer inspects the order to determine if there is any context explaining why the results are missing. If no explanatory context is found, the reviewer forwards the support request email to the appropriate personnel to resolve. Once the support team either adds the HLA results or provides context explaining why they are missing, the reviewer deletes the HLA results missing flag from the internal note and completes the order.
no MATCH date: This support request is issued if MATCH has not been run. The reviewer runs MATCH. If it completes successfully, the reviewer deletes the no MATCH date flag from the internal note, reruns the auto reviewer 240, and completes the order if no additional issues are identified. If auto reviewer 240 does not complete successfully, the reviewer forwards the support request email to the appropriate personnel for completion. Once completed, the reviewer deletes the no MATCH date flag from the internal note, reruns the auto reviewer 240, and completes the order if no additional issues are identified.
duplicated evidence: This support request is issued if a therapy evidence statement includes duplicated text. The reviewer inspects the output files 242 to determine which text is duplicated and determines whether the duplicated text is valid or invalid. If the duplicated text is invalid, the reviewer forwards the support request email to the appropriate personnel for duplication removal. Once the duplication is removed, the reviewer deletes the duplicated evidence flag from the internal note 244 and completes the order. An example of invalid duplicated text is when a full sentence or part of a sentence is repeated.
   Valid duplication example: |P-24-01706|duplicated evidence: neratinib+/−trastuzumab+/−fulvestrant
   This duplication is valid because the string “neratinib+/−trastuzumab+/−fulvestrant [‘0% vs’]” appears validly in two separate sentences.
   Invalid duplication example: duplicated evidence:erdafitinib [‘In a Phase II trial (BCL2001) that supported FDA approval, Balversa (erdafitinib) treatment results in an objective response rate of 40% (40/99, 3 complete response, 37 partial response) and a disease control rate of 80% in patients with metastatic or unresectable urothelial carcinoma harboring FGFR alterations (PMID: 31340094; NCT02365597).’] HOLD.
   This duplication is invalid because a full sentence is repeated.
trial distances missing: This support request is issued if the matched clinical trials do not have associated distances from the patient. The reviewer inspects the order in the report database 223 to determine if there is any context that makes the missing distances valid. If context is identified, the reviewer updates the internal note field to include that context. If no context is identified, the reviewer forwards the support request email to laboratory personnel to update the order such that it can determine trial distances. The reviewer reruns MATCH, reruns the auto reviewer 240, and completes the order assuming no additional issues are identified. An example of context that makes missing distances valid include if the provider facility appears to be from a country outside of the United States. In those cases, trial distances are not necessary. Another example of context that does not make the missing distances value, but does not require forwarding the support request team, includes if MATCH is not run. In that case, the reviewer runs MATCH, reruns the auto reviewer 240, and completes the order assuming no additional issues are identified.
null zip codes: This support request is issued if the ordering_zip and patient_zip fields are null. The reviewer inspects the order homepage in the data store 217 and determines if the null fields are intended (If the order is international the fields are sometimes left null). If the null fields are not intended, the reviewer forwards the support request to OmniSeq for review.
ordering state mismatch: This support request is issued if the state corresponding to the ordering_zip field does not match during cross-referencing. The reviewer forwards the support request to the order assigning team and waits for feedback. The assigning team either updates the fields such that they are equal or indicates that the order can be completed as is. Once the fields are updated or it are indicated that the order can be completed as is, the reviewer deletes the ordering state mismatch flag from the internal note and enters “complete” (assuming this is the only outstanding issue on the order).
mcode mismatch: This support request is issued if the mcode_path field does not match during cross-referencing. The reviewer forwards the support request to the appropriate personnel to update the fields such that they are equal. Once the fields are updated, the reviewer deletes the mcode mismatch flag from the internal note and completes the order. If the field is updated, then the reviewer re-runs MATCH before completing the order.
null report disease: This support request is issued if the report disease name is null. The reviewer forwards the support request to the appropriate personnel who adds the report disease name. Once the report disease name is present, the reviewer re-runs MATCH, reruns the auto reviewer 240, and completes the order if no additional issues are identified.
null mcode: This support request is issued if the mcode field is null. The reviewer forwards the support request to the appropriate personnel and waits for feedback. Once the support team member updates the mcode field, the reviewer deletes the null mcode flag from the internal note, re-runs MATCH, reruns the auto reviewer 240, and completes the order if no additional issues are identified.
disease invalid: This support request is issued if the disease field is invalid. The reviewer forwards the support request to the appropriate personnel and waits for feedback. Once the support team member updates the disease field, the reviewer deletes the disease invalid flag from the internal note, re-runs MATCH, reruns the auto reviewer 240, and completes the order if no additional issues are identified.
mcode-report disease name mismatch: This support request is issued if the report disease name does not match during cross-referencing. The reviewer forwards the support request to the appropriate personnel and waits for feedback. Once the support team member updates the report_disease_name field or the mcode_path field, the reviewer deletes the mcode-report disease name mismatch flag from the internal note, re-runs MATCH, reruns the auto reviewer 240, and completes the order assuming no additional issues are identified.
report disease name is not a solid tumor: This support request is issued if the report_disease_name field implies a non-solid tumor cancer type. The reviewer forwards the support request to the appropriate personnel and waits for feedback. The support team member either updates the report_disease_name field or indicates that the field is valid as is. If the field is valid as is, the reviewer deletes the report disease name is not a solid tumor flag and completes the order. If the support team member updates the field, the reviewer deletes the report disease name is not a solid tumor flag, re-runs MATCH, reruns the auto reviewer 240, and completes the order assuming no additional issues are identified.
PD-L1 IHC+RNA: This support request is issued if there are one or more clinical trials associated with a combination of PD-L1 by IHC and PD-L1 by RNA. The reviewer forwards the support request to the appropriate personnel and waits for them to remove the combination clinical trials from the report. The reviewer deletes the PD-L1 IHC+RNA flag from the internal note and completes the order.
incorrect PD-L1 assay: This support request is issued if the PD-L1 assay attached to the order is not correct given the order report_disease_name. The reviewer forwards the support request to the appropriate personnel, waits for the support team member to update the assay, and for a pathologist to score the new assay. Once the new assay has been scored, the reviewer deletes the incorrect PD-L1 assay flag from the internal note and updates the review log 246 to “complete” (assuming this was the only outstanding issue on the order).
PD-L1 static fields missing: This support request is issued if any of the static text fields associated with the PD-L1 results are missing. The reviewer forwards the support request to the appropriate personnel and waits for feedback. Once the support team member has added the missing text, the reviewer deletes the PD-L1 static fields missing flag from the internal note and completes the order.
PD-L1 score is not an integer: This support request is issued if the PD-L1 score does not take an integer value. The reviewer forwards the support request to the appropriate personnel and waits for feedback. Once the support team member provides the correct PD-L1 score value, the reviewer enters that score in the PD-L1 section, deletes the PD-L1 score is not an integer flag from the internal note, re-runs MATCH, reruns the auto reviewer 240, and completes the order assuming no additional issues are identified.
null PD-L1 assay: This support request is issued if there is no PD-L1 assay attached to the report. The reviewer forwards the support request to the appropriate personnel and waits for feedback. Once the support team member adds a PD-L1 assay and a pathologist scores the assay, the reviewer deletes the null PD-L1 assay flag from the internal note, re-runs MATCH, reruns the auto reviewer 240, and completes the order assuming no additional issues are identified.
PD-L1 assay-scoring method mismatch: This support request is issued if the scoring method selected on the PD-L1 report is incorrect given the associated PD-L1 assay. The reviewer forwards the support request to the appropriate personnel and waits for feedback. Once the support team member updates the scoring method, the reviewer deletes the PD-L1 assay-scoring method mismatch flag from the internal note, re-runs MATCH, reruns the auto reviewer 240, and completes the order assuming no additional issues are identified.
patient name inconsistent capitalization: This support request is issued if the patient's name has inconsistent capitalization. The reviewer forwards the support request to the appropriate personnel and waits for feedback. The support team member either updates the patient's name or indicates that it is valid as is. Once the support team member either updates the patient's name or indicates that it is valid as is, the reviewer deletes the patient name inconsistent capitalization flag from the internal note and completes the order. Consistent capitalization is defined as either fully capitalized (e.g., JOHN SMITH) or title-case capitalized (e.g., John Smith).
invalid character in patient name: This support request is issued if the patient name includes an invalid character. The reviewer forwards the support request to the appropriate personnel and waits for feedback. The support team member either updates the patient's name or indicates that it is valid as is. Once the patient name is either updated or indicated as valid, the reviewer deletes the invalid character in patient name flag from the internal note and completes the order. Invalid characters are any non-alpha characters with the exclusion of “-”.
invalid DOB: This support request is issued if the patient date of birth is invalid. The reviewer forwards the support request to the appropriate personnel and waits for feedback. Once the support team member updates the date of birth, the reviewer deletes the invalid DOB flag from the internal note, re-runs MATCH, reruns the auto reviewer 240, and completes the order assuming no additional issues are identified.
inconsistent capitalization in ordering provider name: This support request is issued if there is inconsistent capitalization in the ordering provider name. The reviewer forwards the support request to the appropriate personnel and waits for feedback. The support team member either updates the ordering provider name or indicates that it is valid as is. Once the support team member has either updated the ordering provider name or indicated that it is valid as is, the reviewer deletes the ordering provider name inconsistent capitalization flag from the internal note and completes the order. Consistent capitalization is defined as either fully capitalized (e.g., JOHN SMITH) or title-case capitalized (e.g., John Smith). Title-case capitalization will count as inconsistent for the provider suffix component of the ordering provider name. Meaning, “MD” would be considered consistent, but “Md” would not.
invalid specimen collection date: This support request is issued if the specimen collection date is invalid. The reviewer forwards the support request to the appropriate personnel and waits for feedback. Once the specimen collection date is updated, the reviewer deletes the invalid specimen collection date flag from the internal note and completes the order. Non-limiting examples of invalid specimen collection date are dates prior to patient date of birth, dates prior to specimen received date, and dates prior to 1900.
duplicated sample label: This support request is issued if multiple samples associated with the order have the same label. The reviewer forwards the support request to the assigning team and waits for feedback. The assigning team either updates the labels or indicates that the order can be completed as is. Once the fields are updated or the assigning team indicates that the order can be completed as is, the reviewer completes the order.
null sample received date: This support request is issued if the sample_received_date field is null. The reviewer forwards the support request to the appropriate personnel and waits for feedback. Once the support team member adds the missing sample received date, the reviewer deletes the null sample received date flag from the internal note and completes the order.
null sample type: This support request is issued if one or more sample_type fields are null. The reviewer forwards the support request to the appropriate personnel and waits for feedback. Once the support team member adds the missing sample type(s), the reviewer deletes the null sample type flag from the internal note and completes the order.
zero testing samples: This support request is issued if none of the samples associated with the order have a sample type equal to testing. The reviewer forwards the support request to the appropriate personnel and waits for feedback. Once the support team member updates at least one of the samples to have a sample type of testing, the reviewer deletes the zero testing samples flag from the internal note and completes the order.
null sample purpose: This support request is issued if any of the samples associated with the order have a null sample purpose field. The reviewer forwards the support request to the appropriate personnel and waits for feedback. Once the support team member updates the sample purpose field(s), the reviewer deletes the null sample purpose flag from the internal note and completes the order.
null sample quantity: This support request is issued if any of the samples associate with the order have a null sample_quantity field. The reviewer forwards the support request to the appropriate personnel and waits for feedback. Once the support team member updates the sample quantity field(s), the reviewer deletes the null sample quantity flag from the internal note and completes the order.
null tissue site: This support request is issued if the tissue_site field is null. The reviewer forwards the support request to the appropriate personnel and waits for feedback. Once the support team member updates the issue_site field, the reviewer deletes the null tissue site flag from the internal note and completes the order.
null tumor origin: This support request is issued if the tumor_origin field is null. The reviewer forwards the support request to the appropriate personnel and waits for feedback. Once the support team member updates the tumor_origin field, the reviewer deletes the null tumor origin flag from the internal note and completes the order.
null necrosis: This support request is issued if the necrosis field is null. The reviewer forwards the support request to the appropriate personnel and waits for feedback. Once the support team member updates the necrosis field, the reviewer deletes the null necrosis flag from the internal note and completes the order.
null percent tumor nuclei: This support request is issued if the percent_tumor_nuclei field is null. The reviewer forwards the support request to the appropriate personnel and waits for feedback. Once the support team member updates the percent tumor nuclei field, the reviewer deletes the null percent tumor nuclei flag from the internal note and completes the order.
null neoplastic cells: This support request is issued if the neoplastic_cells field is null. The reviewer forwards the support request to the appropriate personnel and waits for feedback. Once the support team member updates the neoplastic cells field, the reviewer deletes the null neoplastic cells flag from the internal note and completes the order. Examples of invalid values are “Cancer”, “Unspecified”, and Null. Examples of invalid date of birth are dates after to the specimen collection date and dates prior to 1900.
null pathology report ID: This support request is issued if the pathology_report_id field of the report is null. The reviewer forwards the support request to the appropriate personnel and waits for feedback. Once the support team member updates the pathology_report_id field, the reviewer deletes the null pathology report ID flag from the internal note and completes the order.
ICD information duplicated: This support request is issued if the secondary ICD fields contain the same information as the primary ICD fields. The reviewer forwards the support request to the appropriate personnel and waits for feedback. Once the support team member removes the duplicated information, the reviewer deletes the ICD information duplicated flag from the internal note and completes the order.
null ICD: This support request is issued if the primary ICD fields are null. The reviewer forwards the support request to the appropriate personnel and waits for feedback. Once the support team member enters the ICD information, the reviewer deletes the null ICD flag from the internal note and completes the order.

Once the support team member corrects the issue(s), the reviewer manually updates the internal notes 244 and updates the review log 246 to “complete”. The internal notes 244 are used by the review team to document changes made to a report and why those changes are made. After correction, a notification 248 such as an email is sent (with corrected output attachments) summarizing the status and internal notes of the reviewed orders to the appropriate personnel and order assigners.

Once corrections are made to an order, the auto reviewer 240 may be rerun again to confirm that there are no remaining errors. In the event errors remain, one or more additional rounds of manual review, correction, and log updating are performed iteratively until there are no remaining issues. When no remaining issues are detected, the auto reviewer system 200 has essentially generated an error free order (the series of data entries in data store 217 corresponding to the order are free of errors). A final report 250 may then be generated based on the error free order. The final report 250 may be generated based on the error free order by the auto reviewer system 200 itself or by a different software program such as a part of the LIMS or one or more workbenches. For example, once a status of an order in the review log 246 is changed to “complete,” a user may initiate a final report 250 to be generated via the auto reviewer system 200 itself or by a different software program such as a part of the LIMS or one or more workbenches. Alternatively, once a status of an order in the review log 246 is changed to “complete,” an automated system such as a report monitor may initiate a final report 250 to be generated via the auto reviewer system 200 itself or by a different software program such as a part of the LIMS or one or more workbenches.

The final report 250 is a document that presents relevant information from the error free order to a user in an understandable format. More specifically, final report 250 summarizes the development of different activities based on task progress, goals, and objectives of the user. The content displayed in final report 250 is dependent on the organization and user reviewing the report. For example, a report provided to a healthcare provider of a patient may include patient information (e.g., patient identifications, demographics, etc.) a summary of various lab testing/assays (e.g., blood work, genetic tests, cancer screenings, etc.), medications and treatment plans, therapy considerations, notes on the various lab testing/assays including insights and possible diagnostic interpretations, and the like.

FIG. 3 shows an example of a final report (e.g., a health report) that can be generated after the auto reviewer system 200 terminates. The final report presents medical information of interest to a healthcare provider to aid in the diagnosis of a disease or developing a treatment plan for a patient. More specifically, the final report summarizes information about the patient, what sample was collected and tested, and the various results and conclusions from the completion of those tests. As shown in FIG. 3, the final report provides the results from a “Marker Findings” test broken down into positive genomic variants, signatures, immune markers, and pertinent negative genomic variant fields. Based on these results, a therapy consideration is included in the report for consideration by the reviewing healthcare provider.

Therapy consideration can indicate whether a patient, based on their genomic and immunogenic profiles, and tumor type, is a potential candidate for participation in a clinical trial, receiving certain treatment therapies with FDA approval or recommended in NCCN guidelines, etc. In some instances, the therapy consideration section also displays information related to clinical trials for other tumor types in FDA labels or NCCN guidelines that are matches for the patient. Other notations that may be found in therapy considerations include treatment suggestions. For example, a patient's tumor type can be predictive of which therapies are more likely to be beneficial versus therapies the tumor is more likely to be resistant or have decreased responsivity to. Also included in this report section can be information pertaining to genomic variants (e.g., functional, pathogenic, and/or deleterious effects) that are identified but do not have a matching therapy (e.g., reported from the matching algorithm described above). Comments included on a report can relate to comments from the pathologist, testing, potential germline/somatic variants, and the like.

Although the above description of auto reviewer system 200 describes manual processes for running the auto reviewer 240, for corrections of inconsistent data or errors in the data, and communication of some notifications for detected defects to the appropriate team, laboratory personnel, or administration personnel, either one or more of these processes may be performed automatically without departing from the spirit and scope of the present invention using one or more software programs. For example, auto reviewer 240 can be programed to automatically detect when an order/report is ready for review and will thus automatically execute the program. Further, as part of the program execution, one or more modules of the review and report subsystem 215 can also be programmed to automatically correct inconsistent data or errors in the data and generate and forward notifications for detected defects to the appropriate team, laboratory personnel, or administration personnel.

As previously described, order or report review is traditionally performed primarily by humans who are only able to reasonably check 10-20 rules maximum per report. Integration of the auto reviewing system 200 significantly increases the number of rules (e.g., at least 150 rules) that are checked per order and automate tasks previously performed manually, such as confirming every field in an order includes a data value, no spelling errors in patient name, etc. Further, where it would normally take a human 6-8 hours to review 100 forms, the auto reviewer system 200 processes the same number of reports in less than 10 minutes. More preferably, the auto reviewer system 200 processes the same number of reports in 5 minutes or less, for example in 30 seconds, 1 minute, 2 minutes, 3 minutes, 4 minutes, or 5 minutes.

### Review of Orders Using an Auto Reviewer System

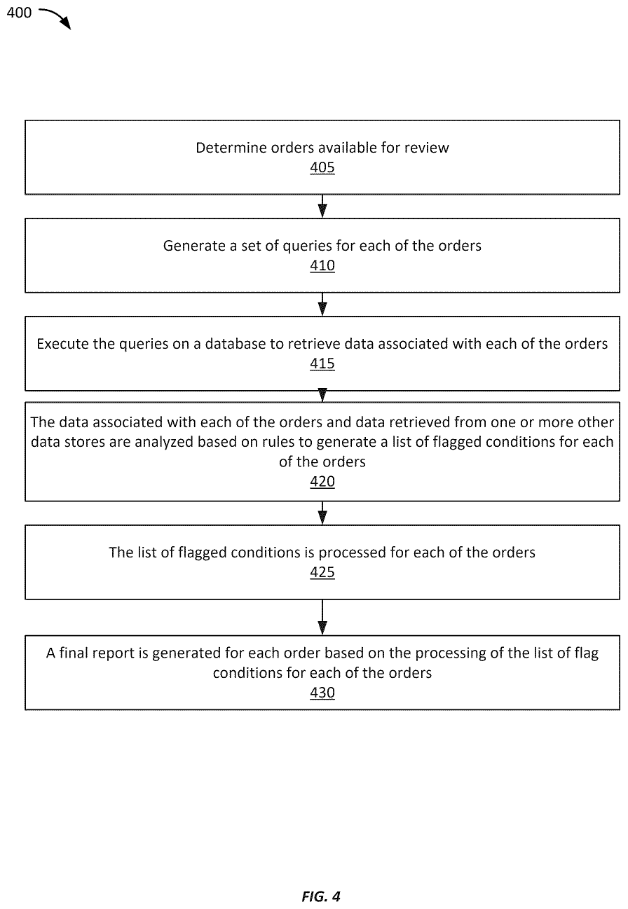

FIG. 4 is a flowchart illustrating process 400 for reviewing orders using an auto reviewer system. The processing depicted in FIG. 4 may be implemented in software (e.g., code, instructions, program) executed by one or more processing units (e.g., processors, cores) of the respective systems, hardware, or combinations thereof (e.g., the intelligent selection machine). The software may be stored on a non-transitory storage medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the steps may be performed in some different orders, or some steps may also be performed in parallel. In certain embodiments, such as in the embodiments depicted in FIGS. 1 and 2, the processing depicted in FIG. 4 may be performed by an auto reviewer system, as described with respect to FIGS. 1 and 2.

At step 405, orders that are available for review are determined based at least on a status of each of the orders. Each of the orders comprise a series of data entries in a table, the status of each of the orders is associated with at least one of the data entries, a unique identifier for each of the orders is associated with at least one of the data entries, and the series of data entries are stored in a production level data store. In some instances, one or more orders such as one order, two orders, or batches of orders (e.g., comprising more than two orders) are available for review.

At step 410, a set of queries for each of the orders is generated based on one or more query programming languages. The set of queries for each of the orders comprise the unique identifier for the associated order.

At step 415, the set of queries for each of the orders are executed on a database to retrieve data associated with each of the orders based on the unique identifier for each of the associated orders. The data is replicated and maintained consistent with the production level data store that obtains at least some of the data from a laboratory instrument, a laboratory assay, a laboratory workstation, or any combination thereof.

In some aspects, process 400 further includes: extracting data from the production level data store, where the data is in non-standardized data files and comprises data expected to appear in the orders; transforming the data in the non-standardized data files into data in a standardized format using one or more transformation algorithms; and storing the data in the standardized form in the database. The set of queries for each of the orders are executed on the data in the standardized format in the database to retrieve the data associated with each of the orders.

In some aspects, the set of queries for each of the orders are executed on the database and the one or more other data stores to retrieve the data associated with each of the orders and the data retrieved from the one or more other data stores based on the unique identifier for each of the associated orders.

At step 420, for each of the orders, the data associated with each of the orders and data retrieved from one or more other data stores are analyzed based on rules defined in a configuration file. The analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied.

At step 425, for each of the orders, the list of flagged conditions are processed. The processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof.

In some aspects, the processing comprises correcting the information within the series of data entries associated with the order in the production level data store. The correcting comprises transmitting the list of flagged conditions associated with the one or more of the orders to a client device, and correcting, using the client device, the information within the series of data entries associated with the one or more of the orders in the production level data store based on the list of flagged conditions associated with the one or more of the orders.

In some aspects, the processing comprises communicating the internal notes pertaining to the information within the series of data entries associated with the order to the production level data store. The communicating comprises generating the internal notes pertaining to the information within the series of data entries associated with the order based on the list of flagged conditions associated with the one or more of the orders, and transmitting one or more write requests to the production level data store for an internal notes field associated with the one or more orders.

In some aspects, the processing comprises updating the status of one or more of the orders in the review log. The updating comprises writing the status of one or more of the orders in the review log based on the list of flagged conditions associated with the one or more of the orders, and transmitting the review log to a client device.

In some aspects, the processing comprises sending one or more notifications to one or more users concerning one or more of the flagged conditions associated with one or more orders. The sending comprises generating one or more notification messages for the one or more of the flagged conditions associated with the one or more orders, and transmitting the one or more notification messages to one or more end points associated with the one or more users.

At step 430, for each of the orders, a final report comprising a summary of the series of data entries is generated based on the series of data entries in the table and the processing the list of flagged conditions.

### Additional Considerations

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed:

1. A computer-implemented method comprising:
determining that orders are available for review based at least on a status of each of the orders, wherein each of the orders comprise a series of data entries in a table, the status of each of the orders is associated with at least one of the data entries, a unique identifier for each of the orders is associated with at least one of the data entries, and the series of data entries are stored in a production level data store;
generating a set of queries for each of the orders based on one or more query programming languages, wherein the set of queries for each of the orders comprise the unique identifier for the associated order;
executing the set of queries for each of the orders on a database to retrieve data associated with each of the orders based on the unique identifier for each of the associated orders, wherein the data is replicated and maintained consistent with the production level data store that obtains at least some of the data from a laboratory instrument, a laboratory assay, a laboratory workstation, or any combination thereof;
for each of the orders, analyzing the data associated with each of the orders and data retrieved from one or more other data stores based on rules defined in a configuration file, wherein the analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied;
for each of the orders, processing the list of flagged conditions, wherein the processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof; and
for each of the orders, generating, based on the series of data entries in the table and the processing the list of flagged conditions, a final report comprising a summary of the series of data entries.

2. The computer-implemented method of claim 1, further comprising:
extracting data from the production level data store, wherein the data is in non-standardized data files and comprises data expected to appear in the orders;
transforming the data in the non-standardized data files into data in a standardized format using one or more transformation algorithms; and
storing the data in the standardized form in the database,
wherein the set of queries for each of the orders are executed on the data in the standardized format in the database to retrieve the data associated with each of the orders.

3. The computer-implemented method of claim 1, wherein the set of queries for each of the orders are executed on the database and the one or more other data stores to retrieve the data associated with each of the orders and the data retrieved from the one or more other data stores based on the unique identifier for each of the associated orders.

4. The computer-implemented method of claim 1, wherein the processing comprises correcting the information within the series of data entries associated with the order in the production level data store, and wherein the correcting comprises transmitting the list of flagged conditions associated with the one or more of the orders to a client device, and correcting, using the client device, the information within the series of data entries associated with the one or more of the orders in the production level data store based on the list of flagged conditions associated with the one or more of the orders.

5. The computer-implemented method of claim 1, wherein the processing comprises communicating the internal notes pertaining to the information within the series of data entries associated with the order to the production level data store, and wherein the communicating comprises generating the internal notes pertaining to the information within the series of data entries associated with the order based on the list of flagged conditions associated with the one or more of the orders, and transmitting one or more write requests to the production level data store for an internal notes field associated with the one or more orders.

6. The computer-implemented method of claim 1, wherein the processing comprises updating the status of one or more of the orders in the review log, and wherein the updating comprises writing the status of one or more of the orders in the review log based on the list of flagged conditions associated with the one or more of the orders, and transmitting the review log to a client device.

7. The computer-implemented method of claim 1, wherein the processing comprises sending one or more notifications to one or more users concerning one or more of the flagged conditions associated with one or more orders, and wherein the sending comprises generating one or more notification messages for the one or more of the flagged conditions associated with the one or more orders, and transmitting the one or more notification messages to one or more end points associated with the one or more users.

8. A system comprising:
one or more processors; and
one or more computer-readable media storing instructions which, when executed by the one or more processors, cause the system to perform operations comprising:
determining that orders are available for review based at least on a status of each of the orders, wherein each of the orders comprise a series of data entries in a table, the status of each of the orders is associated with at least one of the data entries, a unique identifier for each of the orders is associated with at least one of the data entries, and the series of data entries are stored in a production level data store;
generating a set of queries for each of the orders based on one or more query programming languages, wherein the set of queries for each of the orders comprise the unique identifier for the associated order;
executing the set of queries for each of the orders on a database to retrieve data associated with each of the orders based on the unique identifier for each of the associated orders, wherein the data is replicated and maintained consistent with the production level data store that obtains at least some of the data from a laboratory instrument, a laboratory assay, a laboratory workstation, or any combination thereof;
for each of the orders, analyzing the data associated with each of the orders and data retrieved from one or more other data stores based on rules defined in a configuration file, wherein the analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied;
for each of the orders, processing the list of flagged conditions, wherein the processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof; and
for each of the orders, generating, based on the series of data entries in the table and the processing the list of flagged conditions, a final report comprising a summary of the series of data entries.

9. The system of claim 8, wherein the operations further comprise:
extracting data from the production level data store, wherein the data is in non-standardized data files and comprises data expected to appear in the orders;
transforming the data in the non-standardized data files into data in a standardized format using one or more transformation algorithms; and
storing the data in the standardized form in the database,
wherein the set of queries for each of the orders are executed on the data in the standardized format in the database to retrieve the data associated with each of the orders.

10. The system of claim 8, wherein the set of queries for each of the orders are executed on the database and the one or more other data stores to retrieve the data associated with each of the orders and the data retrieved from the one or more other data stores based on the unique identifier for each of the associated orders.

11. The system of claim 8, wherein the processing comprises correcting the information within the series of data entries associated with the order in the production level data store, and wherein the correcting comprises transmitting the list of flagged conditions associated with the one or more of the orders to a client device, and correcting, using the client device, the information within the series of data entries associated with the one or more of the orders in the production level data store based on the list of flagged conditions associated with the one or more of the orders.

12. The system of claim 8, wherein the processing comprises communicating the internal notes pertaining to the information within the series of data entries associated with the order to the production level data store, and wherein the communicating comprises generating the internal notes pertaining to the information within the series of data entries associated with the order based on the list of flagged conditions associated with the one or more of the orders, and transmitting one or more write requests to the production level data store for an internal notes field associated with the one or more orders.

13. The system of claim 8, wherein the processing comprises updating the status of one or more of the orders in the review log, and wherein the updating comprises writing the status of one or more of the orders in the review log based on the list of flagged conditions associated with the one or more of the orders, and transmitting the review log to a client device.

14. The system of claim 8, wherein the processing comprises sending one or more notifications to one or more users concerning one or more of the flagged conditions associated with one or more orders, and wherein the sending comprises generating one or more notification messages for the one or more of the flagged conditions associated with the one or more orders, and transmitting the one or more notification messages to one or more end points associated with the one or more users.

15. One or more non-transitory computer-readable media storing instructions which, when executed by one or more processors, cause a system to perform operations comprising:
determining that orders are available for review based at least on a status of each of the orders, wherein each of the orders comprise a series of data entries in a table, the status of each of the orders is associated with at least one of the data entries, a unique identifier for each of the orders is associated with at least one of the data entries, and the series of data entries are stored in a production level data store;
generating a set of queries for each of the orders based on one or more query programming languages, wherein the set of queries for each of the orders comprise the unique identifier for the associated order;
executing the set of queries for each of the orders on a database to retrieve data associated with each of the orders based on the unique identifier for each of the associated orders, wherein the data is replicated and maintained consistent with the production level data store that obtains at least some of the data from a laboratory instrument, a laboratory assay, a laboratory workstation, or any combination thereof;
for each of the orders, analyzing the data associated with each of the orders and data retrieved from one or more other data stores based on rules defined in a configuration file, wherein the analyzing comprises (i) executing the rules defined in the configuration file on the data associated with each of the orders and the data retrieved from the one or more other data stores, (ii) determining whether one or more conditions of each of the rules are satisfied by comparing the data associated with each of the orders and the data retrieved from the one or more other data stores, and (iii) generating a list of flagged conditions based on the determining whether the one or more conditions of each of the rules are satisfied;
for each of the orders, processing the list of flagged conditions, wherein the processing comprises: (i) correcting information within the series of data entries associated with an order in the production level data store, (ii) communicating internal notes pertaining to the information within the series of data entries associated with the order to the production level data store, (iii) updating a status of the order in a review log, (iv) sending one or more notifications to a user concerning one or more of the flagged conditions, the status of the order, or both, or (v) any combination thereof; and
for each of the orders, generating, based on the series of data entries in the table and the processing the list of flagged conditions, a final report comprising a summary of the series of data entries.

16. The one or more non-transitory computer-readable media of claim 15, wherein the operations further comprise:
extracting data from the production level data store, wherein the data is in non-standardized data files and comprises data expected to appear in the orders;
transforming the data in the non-standardized data files into data in a standardized format using one or more transformation algorithms; and
storing the data in the standardized form in the database,
wherein the set of queries for each of the orders are executed on the data in the standardized format in the database to retrieve the data associated with each of the orders.

17. The one or more non-transitory computer-readable media of claim 15, wherein the processing comprises correcting the information within the series of data entries associated with the order in the production level data store, and wherein the correcting comprises transmitting the list of flagged conditions associated with the one or more of the orders to a client device, and correcting, using the client device, the information within the series of data entries associated with the one or more of the orders in the production level data store based on the list of flagged conditions associated with the one or more of the orders.

18. The one or more non-transitory computer-readable media of claim 15, wherein the processing comprises communicating the internal notes pertaining to the information within the series of data entries associated with the order to the production level data store, and wherein the communicating comprises generating the internal notes pertaining to the information within the series of data entries associated with the order based on the list of flagged conditions associated with the one or more of the orders, and transmitting one or more write requests to the production level data store for an internal notes field associated with the one or more orders.

19. The one or more non-transitory computer-readable media of claim 15, wherein the processing comprises updating the status of one or more of the orders in the review log, and wherein the updating comprises writing the status of one or more of the orders in the review log based on the list of flagged conditions associated with the one or more of the orders, and transmitting the review log to a client device.

20. The one or more non-transitory computer-readable media of claim 15, wherein the processing comprises sending one or more notifications to one or more users concerning one or more of the flagged conditions associated with one or more orders, and wherein the sending comprises generating one or more notification messages for the one or more of the flagged conditions associated with the one or more orders, and transmitting the one or more notification messages to one or more end points associated with the one or more users.
